SEQUENCE LISTING (35 sequences)

SEQ ID NO: 1 - protein, 990 aa, Homo sapiens [sequence omitted]

SEQ ID NO: 2 - DNA, 2970 bp, Homo sapiens
  source 1..2970: mol_type = other DNA
atggggcccccttccagctcaggcttctatgtgagccacgcagtggccctgctgctggctgggttggtagccgccctcctgctggcgctggccgtactcgccgccttgtacggtcactgcgagcgcgtcccaccgtcggagctgcctggactcagggactcggaagccgagtcttcccctcccctcaggcagaagccgacgccgaccccgaaacccagcagtgcacgcgagctagcggtgacgaccaccccgagcaactggcgacccccggggccctgggaccagctacgcctgccgccctggctcgtgccgctgcactacgatctggagctgtggccgcagctgaggcccgacgagcttccggccgggtctttgcccttcactggccgcgtgaacatcacggtgcgctgcacggtggccacctctcgactgctgctgcatagcctcttccaggactgcgagcgcgccgaggtgcggggacccctttccccgggcactgggaacgccacagtgggccgcgtgcccgtggacgacgtgtggttcgcgctggacacggaatacatggtgctggagctcagtgagcccctgaaacctggtagcagctacgagctgcagcttagcttctcgggcctggtgaaggaagacctcagggagggactcttcctcaacgtctacaccgaccagggcgagcgcagggccctgttagcgtcccagctggaaccaacatttgccaggtatgttttcccttgttttgatgagccagctctgaaggcaacttttaatattacaatgattcatcatccaagttatgtggccctttccaacatgccaaagctaggtcagtctgaaaaagaagatgtgaatggaagcaaatggactgttacaaccttttccactacgccccacatgccaacttacttagtcgcatttgttatatgtgactatgaccacgtcaacagaacagaaaggggcaaggagatacgcatctgggcccggaaagatgcaatcgcaaatggaagtgcagactttgctttgaacatcacaggtcccatcttctcttttctggaggatttgtttaatatcagttactctcttccaaaaacagatataattgccttgtctagttttgacaaccatgcaatggaaaactggggactaatgatatttgatgaatcaggattgttgttggaaccaaaagatcaactgacagaaaaaaagactctgatctcctatgttgtctcccacgagattggacaccagtggtttggaaacttggttaccatgaattggtggaacaatatctggctcaacgagggttttgcatcttattttgagtttgaagtaattaactactttaatcctaaactcccaagaaatgagatctttttttctaacattttacataatatcctcagagaagatcacgccctggtgactagagctgtggccatgaaggtggaaaatttcaaaacaagtgaaatacaggaactctttgacatatttacttacagcaagggagcgtctatggcccggatgctttcttgtttcttgaatgagcatttatttgtcagtgcactcaagtcatatttgaagacattttcctactcaaacgctgagcaagatgatctatggaggcattttcaaatggccatagatgaccagagtacagttattttgccagcaacaataaaaaacataatggacagttggacacaccagagtggttttccagtgatcactttaaatgtgtctactggcgtcatgaaacaggagccattttatcttgaaaacattaaaaatcggactcttctaaccagcaatgacacatggattgtccctattctttggataaaaaatggaactacacaacctttagtctggctagatcaaagcagcaaagtattcccagaaatgcaagtttcagattctgaccatgactgggtgattttgaatttgaatatgactggatattatagagttaattatgataaattaggttggaagaaactaaatcaacaacttgaaaaggatcctaaggcgattcctgttattcacagactgcagttgattgatgatgccttttccttgtctaaaaacaattatattgagattgaaacagcacttgagttaaccaagtaccttgctgaagaagatgaaattatagtatggcatacagtcttggtaaacttggtaaccagggatcttgtttctgaggtgaacatctatgatatatactcattattaaagaggtacctattaaagagacttaatttaatatggaatatttattcaactataattcgtgaaaatgtgttggcattacaagatgactacttagctctaatatcactggaaaaactttttgtaactgcgtgttggttgggccttgaagactgccttcagctgtcaaaagaacttttcgcaaaatgggtggatcatccagaaaatgaaataccttatccaattaaagatgtggttttatgttatggcattgccttgggaagtgataaagagtgggacatcttgttaaatacttacactaatacaacaaacaaagaagaaaagattcaacttgcttatgcaatgagctgcagcaaagacccatggatacttaacagatatatggagtatgccatcagcacatctccattcacttctaatgaaacaaatataattgaggttgtggcttcatctgaagttggccggtatgtcgcaaaagacttcttagtcaacaactggcaagctgtgagtaaaaggtatggaacacaatcattgattaatctaatatatacaatagggagaaccgtaactacagatttacagattgtggagctgcagcagtttttcagtaacatgttggaggaacaccagaggatcagagttcatgccaacttacagacaataaagaatgaaaatctgaaaaacaagaagctaagtgccaggatagctgcgtggctaaggagaaacaca

SEQ ID NO: 3 - protein, 15 aa, synthetic construct
  REGION 1..15
MNLLLILTFVAAVAA

SEQ ID NO: 4 - DNA, 20 bp, synthetic construct
  source 1..20: mol_type = other DNA
tctcagaggagcctggctaa

SEQ ID NO: 5 - DNA, 23 bp, synthetic construct
  source 1..23: mol_type = other DNA
ccagactatccttgacctgatga

SEQ ID NO: 6 - DNA, 20 bp, synthetic construct
  source 1..20: mol_type = other DNA
cagaactgcagggaaacagc

SEQ ID NO: 7 - DNA, 24 bp, synthetic construct
  source 1..24: mol_type = other DNA
tgaataagttccaggtgaaatggc

SEQ ID NO: 8 - DNA, 20 bp, synthetic construct
  source 1..20: mol_type = other DNA
gagaggcagcgaactcatct

SEQ ID NO: 9 - DNA, 20 bp, synthetic construct
  source 1..20: mol_type = other DNA
cttcagctctgacaccgaca

SEQ ID NO: 10 - DNA, 21 bp, synthetic construct
  source 1..21: mol_type = other DNA
aggtggctcctatggacggaa

SEQ ID NO: 11 - DNA, 25 bp, synthetic construct
  source 1..25: mol_type = other DNA
ggcttctcttctgatcctggaattg

SEQ ID NO: 12 - DNA, 19 bp, synthetic construct
  source 1..19: mol_type = other DNA
tggcacccagcacaatgaa

SEQ ID NO: 13 - DNA, 25 bp, synthetic construct
  source 1..25: mol_type = other DNA
ctaagtcatagtccgcctagaagca

SEQ ID NO: 14 - DNA, 20 bp, synthetic construct
  source 1..20: mol_type = other DNA
ggcacacgctatggaaaact

SEQ ID NO: 15 - DNA, 20 bp, synthetic construct
  source 1..20: mol_type = other DNA
cggacatctccatgaccttt

SEQ ID NO: 16 - DNA, 22 bp, synthetic construct
  source 1..22: mol_type = other DNA
tgggctgtgatctgcctcaaac

SEQ ID NO: 17 - DNA, 22 bp, synthetic construct
  source 1..22: mol_type = other DNA
cagccttttggaactggttgcc

SEQ ID NO: 18 - DNA, 21 bp, synthetic construct
  source 1..21: mol_type = other DNA
accatctgaagacagtcctgg

SEQ ID NO: 19 - DNA, 20 bp, synthetic construct
  source 1..20: mol_type = other DNA
ccttggccttcaggtaatgc

SEQ ID NO: 20 - DNA, 23 bp, synthetic construct
  source 1..23: mol_type = other DNA
gagtgtggagaccatcaaggaag

SEQ ID NO: 21 - DNA, 24 bp, synthetic construct
  source 1..24: mol_type = other DNA
tgctttgcgttggacattcaagtc

SEQ ID NO: 22 - DNA, 23 bp, synthetic construct
  source 1..23: mol_type = other DNA
gcagtgtacttcacagagctgtc

SEQ ID NO: 23 - DNA, 22 bp, synthetic construct
  source 1..22: mol_type = other DNA
aagccaaaggctccagtcacca

SEQ ID NO: 24 - DNA, 22 bp, synthetic construct
  source 1..22: mol_type = other DNA
ctggaacaggtgcctaaaggac

SEQ ID NO: 25 - DNA, 22 bp, synthetic construct
  source 1..22: mol_type = other DNA
gtccagtgtcaggttatccacc

SEQ ID NO: 26 - DNA, 24 bp, synthetic construct
  source 1..24: mol_type = other DNA
cactcatcacagcggtactgtaca

SEQ ID NO: 27 - DNA, 19 bp, synthetic construct
  source 1..19: mol_type = other DNA
tccatttgcctcctccatc

SEQ ID NO: 28 - DNA, 20 bp, synthetic construct
  source 1..20: mol_type = other DNA
ttaagcaggcacctctgtcc

SEQ ID NO: 29 - DNA, 20 bp, synthetic construct
  source 1..20: mol_type = other DNA
ctgagcctttgctttcatcc

SEQ ID NO: 30 - DNA, 22 bp, synthetic construct
  source 1..22: mol_type = other DNA
ctcttggtgctggctggtcttt

SEQ ID NO: 31 - DNA, 22 bp, synthetic construct
  source 1..22: mol_type = other DNA
gccagtagatgcgagtttgtgc

SEQ ID NO: 32 - DNA, 24 bp, synthetic construct
  source 1..24: mol_type = other DNA
ccatcagcttgtctgtttcattcc

SEQ ID NO: 33 - DNA, 23 bp, synthetic construct
  source 1..23: mol_type = other DNA
gctgtaatccaaggaatgtggtc

SEQ ID NO: 34 - DNA, 19 bp, synthetic construct
  source 1..19: mol_type = other DNA
aaacaattagacctggctg

SEQ ID NO: 35 - DNA, 19 bp, synthetic construct
  source 1..19: mol_type = other DNA
tcttaccactcaggacttg

(19) United States
(12) Patent Application Publication
(10) Pub. No.: US 2026/0306540 A1
(43) Pub. Date: Oct. 8, 2026

(54) COMPOSITION FOR INDUCING IMMUNOTOLERANCE

(71) Applicant: Hiroshi FUJIWARA, Kyoto-shi, Kyoto (JP)

(72) Inventor: Hiroshi FUJIWARA, Kyoto-shi, Kyoto (JP)

(21) Appl. No.: 18/881,118

(22) PCT Filed: May 23, 2023
(86) PCT No.: PCT/JP2023/019186

§ 371 Date: Jan. 3, 2025

(30) Foreign Application Priority Data
Jul. 14, 2022 (JP) 2022-113357

(51) Int. Cl. A61K 35/17; A61K 38/48; A61K 40/11; A61P 37/06; C12N 5/00; C12N 5/0783; C12Q 1/26; G01N 33/50; G01N 33/68

(52) U.S. Cl. CPC A61K 35/17; A61K 38/4813; A61K 40/11; A61P 37/06; C12N 5/0018; C12N 5/0637; C12Q 1/26; C12Y 304/11; G01N 33/5023; G01N 33/505; G01N 33/6866; C12N 2501/734; C12N 2510/00; G01N 2333/555; G01N 2333/90241; G01N 2800/245; G01N 2800/50; G01N 2800/52

(57) ABSTRACT

The present disclosure includes, inter alia, a composition for inducing immunotolerance, the composition containing laeverin.

### TECHNICAL FIELD

This application claims the benefit of priority of the prior Japanese Patent Application No. 2022-113357, the entire contents of which are incorporated herein by reference.

The present disclosure encompasses a composition for inducing immunotolerance, a method for inducing immunotolerance, a method for treating a cell, a composition for cell transplantation therapy, an antiviral agent, a method for treating a viral disease, a composition for inducing a dendritic cell, a method for preparing a dendritic cell, a method for determining a disease or a risk of onset thereof, and a method for evaluating the effect of a therapeutic agent.

### BACKGROUND ART

Laeverin is a unique glycoprotein that is expressed in an extravillous trophoblast cell (EVT) of the placenta. The EVT is a cell that infiltrates into the maternal endometrium and can infiltrate into the uterine muscle layer without being attacked by a maternal immune cell, and thus is immunologically tolerant. It has been reported that laeverin is involved in the infiltration of the EVT, but the mechanism of establishing the immunotolerance of the EVT has not been clarified yet.

### CITATION LIST

### Patent Literature

Patent Literature 1: JP 2005-160473 A

### Non-Patent Literature

Non-Patent Literature 1: Fujiwara H, et. al., Promoting Roles of Embryonic Signals in Embryo Implantation and Placentation in Cooperation with Endocrine and Immune Systems. Int J Mol Sci. 2020 Mar. 10; 21 (5): 1885. doi: 10.3390/ijms21051885.
Non-Patent Literature 2: Horie A, et. al., Laeverin/aminopeptidase Q induces trophoblast invasion during human early placentation. Hum Reprod. 2012 May; 27 (5): 1267-76. doi: 10.1093/humrep/des068.
Non-Patent Literature 3: Fujiwara H, et. al., Human extravillous trophoblasts express laeverin, a novel protein that belongs to membrane-bound gluzincin metallopeptidases. Biochem Biophys Res Commun. 2004 Jan. 23; 313 (4): 962-8.
Non-Patent Literature 4: Maruyama M, et. al., Histidine 379 of human laeverin/aminopeptidase Q, a nonconserved residue within the exopeptidase motif, defines its distinctive enzymatic properties. J Biol Chem. 2009 Dec. 11; 284 (50): 34692-702. doi: 10.1074/jbc.M109.066712.
Non-Patent Literature 5: Maruyama M, et. al., Laeverin/aminopeptidase Q, a novel bestatin-sensitive leucine aminopeptidase belonging to the M1 family of aminopeptidases. J Biol Chem. 2007 Jul. 13; 282 (28): 20088-96. doi: 10.1074/jbc.M702650200.
Non-Patent Literature 6: Imakawa K, et. al., Interferon-like sequence of ovine trophoblast protein secreted by embryonic trophectoderm. Nature. 1987 Nov. 26-Dec. 2; 330 (6146): 377-9. doi: 10.1038/330377a0.

### SUMMARY OF INVENTION

The present inventor discovered that laeverin induces IDO1 and PD-L1, which are involved in immunotolerance, and induces expression of IFN-β, and a group of ISGs induced thereby. Furthermore, the inventor discovered that pregnant women suffering from a disease such as a hypertensive disorder of pregnancy exhibit, compared with normal pregnant women, low IGS15 expression level, low IDO1 expression level, a low IDO1/ISG15 ratio, and a high proportion of laeverin-positive T cells in their peripheral blood mononuclear cells. The present invention was made based on these findings.

In one aspect, the present disclosure relates to a composition for inducing immunotolerance, comprising laeverin.

In one aspect, the present disclosure relates to a method for inducing immunotolerance, comprising administering an effective amount of laeverin to a subject in need thereof.

In one aspect, the present disclosure relates to a method for treating a cell, comprising culturing, in the presence of laeverin, an immune cell or a cell population containing immune cells.

In one aspect, the present disclosure relates to a composition for cell transplantation therapy, comprising a laeverin-expressing cell(s) comprising a transgene encoding laeverin.

In one aspect, the present disclosure relates to an antiviral agent comprising laeverin.

In one aspect, the present disclosure relates to a method for treating a viral disease, comprising administering an effective amount of laeverin to a subject in need thereof.

In one aspect, the present disclosure relates to a composition for inducing a dendritic cell, comprising laeverin.

In one aspect, the present disclosure relates to a method for preparing a dendritic cell, comprising culturing, in the presence of laeverin, a monocyte or a cell population containing monocytes.

In one aspect, the present disclosure relates to a method for determining a disease or a risk of onset of the disease, comprising culturing, in the presence of laeverin, peripheral blood mononuclear cells collected from a subject; and comparing, to a reference value, ISG15 expression level, IDO1 expression level, or an IDO1/ISG15 ratio in the peripheral blood mononuclear cells after the culturing.

In one aspect, the present disclosure relates to a method for evaluating an effect of a therapeutic agent, comprising culturing, in the presence of laeverin, peripheral blood mononuclear cells collected from a subject with a disease after administration of the therapeutic agent; and comparing, to a reference value, ISG15 expression level, IDO1 expression level, or an IDO1/ISG15 ratio in the peripheral blood mononuclear cells after the culturing.

In one aspect, the present disclosure relates to a method for determining a disease or a risk of onset of the disease, comprising comparing, to a reference value, an amount or a proportion of laeverin-positive T cells in peripheral blood collected from a subject.

In one aspect, the present disclosure relates to a method for evaluating an effect of a therapeutic agent, comprising comparing, to a reference value, an amount or a proportion of laeverin-positive T cells in peripheral blood collected from a subject with a disease after administration of the therapeutic agent.

### BRIEF DESCRIPTION OF DRAWINGS

FIG. 1A shows the results of a gene ontology analysis of genes induced by a recombinant laeverin (rLVRN) in peripheral blood mononuclear cells (PBMC).

FIG. 1B shows the expression of interferon-induced genes (OAS2, IFIT1, IFIT3, and ISG15), IDO1, and PD-L1 in PBMC stimulated with rLVRN. The statistical analysis was performed using a Welch 2-sample t-test. * p<0.05, ** p<0.01.

FIG. 1C shows the results of rLVRN absorption experiments using beads. The statistical analysis was performed using a multiple-comparison test with Bonferroni correction. ** p<0.01.

FIG. 1D shows the induction of the gene in PBMC cultured directly or indirectly with Swan71 cells forced to express LVRN. The statistical analysis was performed using a Welch 2-sample t-test. * p<0.05, ** p<0.01.

FIG. 1E shows the induction of the gene in PBMC by an enzymatic activity-inactivated rLVRN. The statistical analysis was performed using a Welch 2-sample t-test. *p<0.05, ** p<0.01.

FIG. 2A shows an induction of IFN expression in PBMC by rLVRN. The statistical analysis was performed using a Welch 2-sample t-test. ** p<0.01.

FIG. 2B shows the time required to show the effect of gene induction by rLVRN in PBMC. The statistical analysis was performed using a multiple comparison test of ANOVA and Dunnet. * p<0.05, ** p <0.01, *** p<0.001.

FIG. 2C shows a working concentration of IFN-β corresponding to rLVRN (1.5 μg/mL).

FIG. 2D shows the effect of an IFN-β-neutralizing antibody (aIFNb) on gene induction by IFN-β. The statistical analysis was performed using a multiple-comparison test with Bonferroni correction. ** p<0.01.

FIG. 2E shows the effects of an IFN-β-neutralizing antibody (aIFNb) and an IFN receptor type-I neutralizing antibody (aIFNabR) on gene induction by rLVRN. The statistical analysis was performed using a multiple-comparison test with Bonferroni correction. ** p<0.01.

FIG. 2F shows the effect of JAK1,2 inhibitor (Ruxo) on gene induction by rLVRN in PBMC. The statistical analysis was performed using a multiple-comparison test with Bonferroni correction. * p<0.05, ** p<0.01.

FIG. 2G shows a comparison of ISG15- and IDO1-induction effects by IFN-β or rLVRN in PBMC. The statistical analysis was performed using a Welch 2-sample t-test. * p<0.05, ** p<0.01.

FIG. 3A shows the expression of ISG15 and IDO1 after stimulation with rLVRN in PBMCs from normal non-pregnant women (non-preg), normal pregnant women (normal-preg), and women suffering from hypertensive disorders of pregnancy (HDP).

FIG. 3B shows the expression of ISG15 and IDO1 after stimulation with IFNβ or rLVRN in PBMC of normal pregnant women. The statistical analysis was performed using a Welch 2-sample t-test. * p <0.05, ** p<0.01.

FIG. 3C shows the relation between the growth of fetuses and IDO1/ISG15 ratios.

FIG. 3D shows the percentages of LVRN-positive T cells in non-pregnant women, normal pregnant women, women suffering from hypertensive disorders of pregnancy, and pregnant women suffering from an immune disease.

FIG. 4A shows a FACS analysis of ISG15 protein expression in a lymphocyte fraction and a monocyte fraction of PBMC after stimulation with rLVRN.

FIG. 4B shows the staining of cells for the expression of ISG15 protein in CD14-positive monocytes, CD4-positive T lymphocytes, CD8-positive T lymphocytes, and CD19-positive B lymphocytes in PBMC after stimulation with rLVRN.

FIG. 4C shows a FACS analysis of binding or incorporation of a labeled rLVRN to or into a lymphocyte fraction and a monocyte fraction of PBMC.

FIG. 4D shows an analysis, by cell staining, of binding or incorporation of the labeled rLVRN to or into a monocyte fraction of PBMC. FIG. 4E shows the induction of the gene in CD4-positive T lymphocytes and CD8-positive T lymphocytes after stimulation with rLVRN. The statistical analysis was performed using a Welch 2-sample t-test. * p<0.05, ** p<0.01.

FIG. 4F shows a microarray analysis of a group of rLVRN-induced genes in CD14-positive monocyte cells.

FIG. 4G shows the induction of the gene in CD14-positive monocyte cells by rLVRN. The statistical analysis was performed using a Welch 2-sample t-test. * p<0.05, ** p<0.01.

FIG. 4H shows the differentiation induction of CD14-positive monocyte cells into dendritic cells (DC) by rLVRN. The statistical analysis was performed using a Welch 2-sample t-test. * p<0.05, ** p<0.01.

FIG. 4I shows the differentiation induction of CD14-positive monocyte cells into CD11c-positive/CD123-positive DC by rLVRN.

FIG. 5A shows the expression of IDO1 after stimulation with rLVRN in PMA-activated or -inactivated THP-1 cells. The statistical analysis was performed using a Welch 2-sample t-test with Bonferroni correction for multiple test. * p<0.05.

FIG. 5B shows a Western blot analysis of IDO1 protein in PMA-activated THP-1 cells after stimulation with rLVRN. The statistical analysis was performed using a Welch 2-sample t-test. * p<0.05.

FIG. 5C shows the effect of rLVRN on IDO1 induction in PMA-activated THP-1 cells. The statistical analysis was performed using a One-way ANOVA (analysis of variance) and a post hoc Dunnett multiple-comparison test. *** p<0.001.

FIG. 5D shows a Western blot analysis of IDO1 protein in PMA-activated THP-1 cells after stimulation with rLVRN.

FIG. 6 shows changes, caused by rLVRN, in tryptophan level and kynurenine/tryptophan ratio in PMA-activated THP-1 cells. The statistical analysis was performed using a Welch 2-sample t-test with Bonferroni correction for multiple test. * p<0.05.

### DESCRIPTION OF EMBODIMENTS

Unless otherwise specifically defined, terms used in the present disclosure have the same meaning as commonly understood by those skilled in the art of the fields of organic chemistry, medicine, pharmacy, molecular biology, microbiology, and the like. The definitions of some terms used in the present disclosure are described below, and such definitions are herein prioritized over general understanding.

Laeverin (also herein referred to as LVRN) is a glycoprotein identified as a cell surface antigen of an extravillous trophoblast cell (EVT) of the placenta. Laeverin is a cell membrane-binding protein possessing an M1 peptidase activity, and has a membrane-binding site on the N-terminus. The amino acid sequence of laeverin is available from a databank site provided by a public institution.

The representative amino acid sequence of human laeverin is shown in SEQ ID NO: 1, and a nucleotide sequence encoding the amino acid sequence is shown in SEQ ID NO: 2. In SEQ ID NO: 1, amino acids 14 to 36 correspond to the membrane-binding site, and amino acids 415 to 438 correspond to the HEXXH(X)₁₈ E motif that is a Zn binding site, which is an enzyme active site designated as a gluzincin motif. The existence of secretory laeverin has also been reported, and a position between the 64th amino acid and the 65th amino acid in SEQ ID NO: 1 corresponds to the cleavage site (Non-Patent Literature 5). Herein, laeverin may be of membrane-binding type or secretory type. Laeverin may lack peptidase activity and, for example, may harbor a mutation in the enzyme's active site.

In one embodiment, laeverin is a polypeptide comprising or consisting of an amino acid sequence having 80% or more, 85% or more, 90% or more, 95% or more, 96% or more, 97% or more, 98% or more, or 99% or more sequence identity to the amino acid sequence shown in SEQ ID NO: 1. In one embodiment, laeverin is a polypeptide comprising or consisting of an amino acid sequence obtained by modifying 1 to 200, 1 to 150, 1 to 100, 1 to 90, 1 to 80, 1 to 70, 1 to 60, 1 to 50, 1 to 40, 1 to 30, 1 to 20, 1 to 10, 1 to 5, 1 to 3, or 1 to 2 amino acid residues in the amino acid sequence shown in SEQ ID NO: 1. In another embodiment, laeverin is a polypeptide comprising or consisting of the amino acid sequence shown in SEQ ID NO: 1.

In one embodiment, laeverin is a polypeptide comprising or consisting of an amino acid sequence having 80% or more, 85% or more, 90% or more, 95% or more, 96% or more, 97% or more, 98% or more, or 99% or more sequence identity to the amino acid sequence from 65th to 990th amino acids shown in SEQ ID NO: 1. In one embodiment, laeverin is a polypeptide comprising or consisting of an amino acid sequence obtained by modifying 1 to 200, 1 to 150, 1 to 100, 1 to 90, 1 to 80, 1 to 70, 1 to 60, 1 to 50, 1 to 40, 1 to 30, 1 to 20, 1 to 10, 1 to 5, 1 to 3, or 1 to 2 amino acid residues in the amino acid sequence from 65th to 990th amino acids shown in SEQ ID NO: 1. In another embodiment, laeverin is a polypeptide comprising or consisting of the amino acid sequence from 65th to 990th amino acids shown in SEQ ID NO: 1.

In one embodiment, laeverin is a polypeptide comprising or consisting of an amino acid sequence encoded by a nucleotide sequence having 80% or more, 85% or more, 90% or more, 95% or more, 96% or more, 97% or more, 98% or more, or 99% or more sequence identity to the nucleotide sequence shown SEQ ID NO: 2. In one embodiment, laeverin is a polypeptide comprising or consisting of an amino acid sequence encoded by a nucleotide sequence obtained by modifying 1 to 600, 1 to 500, 1 to 400, 1 to 300, 1 to 200, 1 to 100, 1 to 90, 1 to 80, 1 to 70, 1 to 60, 1 to 50, 1 to 40, 1 to 30, 1 to 20, 1 to 10, 1 to 5, 1 to 3, or 1 to 2 nucleotides in the nucleotide sequence shown in SEQ ID NO: 2. In another embodiment, laeverin is a polypeptide comprising or consisting of an amino acid sequence encoded by the nucleotide sequence shown in SEQ ID NO: 2.

In one embodiment, laeverin is a polypeptide comprising or consisting of an amino acid sequence encoded by a nucleotide sequence with 80% or more, 85% or more, 90% or more, 95% or more, 96% or more, 97% or more, 98% or more, or 99% or more sequence identity to a nucleotide sequence from 193rd to 2970th nucleotides shown in SEQ ID NO: 2. In one embodiment, laeverin is a polypeptide comprising a nucleotide sequence obtained by modifying 1 to 600, 1 to 500, 1 to 400, 1 to 300, 1 to 200, 1 to 100, 1 to 90, 1 to 80, 1 to 70, 1 to 60, 1 to 50, 1 to 40, 1 to 30, 1 to 20, 1 to 10, 1 to 5, 1 to 3, or 1 to 2 nucleotides in the nucleotide sequence from the 193rd to the 2970th nucleotides shown in SEQ ID NO: 2, or consisting of the amino acid sequence. In another embodiment, laeverin is a polypeptide comprising or consisting of an amino acid sequence encoded by the nucleotide sequence from the 193rd to the 2970th nucleotides shown in SEQ ID NO: 2.

In one embodiment, laeverin has an amino acid length of 700 to 1200, 800 to 1100, or 900 to 1000 amino acids.

Herein, modification of an amino acid(s) or a nucleotide(s) encompasses deletion, substitution, insertion, and addition of an amino acid(s) or a nucleotide(s), or a combination thereof. The modification of an amino acid(s) or a nucleotide(s) may be conducted in any position(s).

Herein, sequence identity is the ratio of amino acids or nucleotides identical between two sequences aligned in an optimal state (i.e., the maximum identity) over the entire regions of the sequences to be compared. In the optimal alignment of two sequences, there may be insertions or deletions (e.g., gaps). The sequence identity can be calculated by using a program, such as FASTA, BLAST, or CLUSTAL W, provided by a public database (e.g., DDBJ (http://www.ddbj.nig.ac.jp)). Alternatively, the sequence identity can be obtained by using commercially available sequence analysis software (e.g., Vector NTI® software, or GENETYX® ver. 12).

In one embodiment, laeverin is a polypeptide functionally equivalent to the polypeptide consisting of the amino acid sequence shown in SEQ ID NO: 1 or the amino acid sequence consisting of 65th to 990th amino acids shown in SEQ ID NO: 1. Being functionally equivalent to the polypeptide consisting of the amino acid sequence shown in SEQ ID NO: 1 or the amino acid sequence consisting of 65th to 990th amino acids shown in SEQ ID NO: 1 means having equivalent activity to the polypeptide. In one embodiment, the polypeptide functionally equivalent to the polypeptide consisting of the amino acid sequence shown in SEQ ID NO: 1 or the amino acid sequence consisting of 65th to 990th amino acids shown in SEQ ID NO: 1 has one or more activities selected from immunotolerance-inducing activity, antiviral activity, and dendritic cell-inducing activity.

Laeverin can be produced by any of conventional methods employed for polypeptide production, such as genetic modification and chemical synthesis. For example, in employing genetic modification, laeverin can be produced by introducing a vector containing a sequence encoding laeverin into a host cell to obtain a transformant expressing laeverin. Examples of vector types include plasmid, phage, and viral vectors. The viral vector encompasses a retroviral vector, a lentiviral vector, an adenoviral vector, an adeno-associated viral vector, and a baculoviral vector. The host cell may be E. coli, yeast, insect cells, animal cells, plant cells, or the like. The vector usually contains, in addition to the sequence encoding laeverin, a regulatory sequence, such as a promoter, to adjust expression. The regulatory sequence is selected so as to be suitable for expression in the host cell. The vector may further contain another element, such as a sequence encoding a selectable marker for selecting a transformant, or a sequence encoding a tag such as a histidine (His) tag, or a glutathione S-transferase (GST) tag. The vector can be introduced into the host cell by electroporation, lipofection, calcium phosphate method, DEAE-dextran method, or the like. Laeverin can be obtained from the resulting transformant, or a culture fluid thereof. Laeverin can be isolated and purified using any of the methods commonly employed for polypeptide isolation and purification, such as ammonium sulfate precipitation, gel permeation chromatography, ion exchange chromatography, or affinity chromatography.

Laeverin may be a fusion polypeptide having a tag or another sequence added thereto, or may be bonded to a carrier such as beads. Alternatively, laeverin may be incorporated into an artificial lipid bilayer membrane (https://bio-med.jp/projects/hanayama/; Drug Delivery System 35-1, p. 35-46, 2020).

It has been suggested that laeverin induces IDO1 (indoleamine 2,3-dioxygenase 1) and PD-L1 (programmed death-ligand 1), which are involved in immunotolerance in peripheral blood mononuclear cells, and that laeverin induces IDO1 more strongly in pregnant subjects, who need immunotolerance to their embryo or fetus, compared to non-pregnant subjects. IDO1 metabolizes tryptophan, which is an amino acid essential for cell survival, to convert it to kynurenine, a metabolite that has an immunosuppressive effect. Accordingly, when IDO1 expression increases in an immune cell, immunosuppression is induced. In addition, when PD-1 expressing in an activated T cell binds to PD-L1 or PD-L2 expressing in a cancer cell or an antigen presenting cell, the T cell activation is suppressed to cause immune evasion of the cancer cell (Song X, Si Q, Qi R, Liu W, Li M, Guo M, Wei L, Yao Z. Indoleamine 2,3-Dioxygenase 1: A Promising Therapeutic Target in Malignant Tumor. Front Immunol. 2021 Dec. 23; 12:800630. doi: 10.3389/fimmu.2021.800630. PMID: 35003126; PMCID: PMC8733291; Kythreotou A, Siddique A, Mauri F A, Bower M, Pinato D J. PD-L1. J Clin Pathol. 2018 March; 71 (3): 189-194. doi: 10.1136/jclinpath-2017-204853. Epub 2017 Nov. 2. PMID: 29097600). Therefore, laeverin, capable of inducing IDO1 and PD-L, can be used for inducing immunotolerance.

Herein, immunotolerance refers to a state in which a specific immune response to a specific antigen does not occur or is diminished. In one embodiment, laeverin is used for inducing immunotolerance in a subject. In another embodiment, laeverin is used for inducing immunotolerance in a cell. For example, laeverin can be used in the treatment of a subject in need of immunotolerance induction, or the treatment of a cell in need of immunotolerance induction.

In one embodiment, laeverin is administered to a patient with an autoimmune disease. Examples of autoimmune diseases include systemic lupus erythematosus (SLE), rheumatoid arthritis, Sjogren's syndrome, Hashimoto's disease, type 1 diabetes, autoimmune myositis, systemic scleroderma, glomerulonephritis, and Graves' disease. In one aspect, the present disclosure provides a composition for treating an autoimmune disease, comprising laeverin.

In one embodiment, laeverin is administered to a recipient of organ transplantation. In organ transplantation, an immunosuppressant is used for suppressing rejection to the transplanted organ caused by the immune function of the recipient, but since immune functions necessary for maintaining homeostasis of the living body are also suppressed thereby, there is a concern that a risk of an infectious disease may increase, or a risk of cancer onset may increase because of the suppression of cancer immunity. When immunotolerance is induced in a recipient of organ transplantation, the withdrawal of immunosuppressants can be expected.

In one embodiment, laeverin is administered into the uterus prior to embryo transfer after in vitro fertilization. Laeverin is a molecule involved in the induction of immunotolerance to a fetus, and the immune environment of the endometrium can be placed in a state suitable for implantation by administering laeverin, and thus, the implantation rate in the embryo transfer can be improved.

In one embodiment, laeverin is used in treatment of an immune cell, or a cell population containing immune cells. Examples of immune cells include T cells (including CD4-positive T cells and CD8-positive T cells), B cells, NK cells, monocytes, macrophages, dendritic cells, and combinations thereof; the immune cell is preferably monocytes. An example of the cell population containing immune cells includes peripheral blood mononuclear cells. Peripheral blood mononuclear cells can be isolated from peripheral blood by a general method, such as density gradient centrifugation. The immune cells, or the cell population containing immune cells, can be immune cells collected from a subject in need of immunotolerance induction, or a cell population containing those immune cells. For example, immune cells or a cell population containing immune cells may be collected from a subject in need of immunotolerance induction, be treated with laeverin, and be returned to the subject. Examples of subjects in need of immunotolerance induction include patients with autoimmune disease and organ transplant recipients.

Regarding the in-vitro fertilization, it has been reported that the implantation rate is improved by autoimmune transplantation therapy in which embryo transfer is performed after activating autoimmune cells taken out of the body with an embryonic signal of human chorionic gonadotropin (HCG), and administering the resultant into the uterus, and the therapeutic effect has been confirmed by a large number of replication studies (Yoshioka et al., Hum Reproduction, vol. 21, No. 12, pp. 3290-3294, 2006). Laeverin can be used as an embryonic signal replaceable with HCG in this autoimmune transplantation therapy. Therefore, the subject in need of immunotolerance induction encompasses a recipient of embryo transfer.

The treatment of a cell or a cell population with laeverin can be performed by culturing the cell or the cell population in the presence of laeverin. As the medium, any medium usually used in culturing an animal cell can be used, and for example, RPMI1640, MEM, IMDM, DMEM, and the like can be used. Laeverin can be added to the medium at a final concentration of, for example, 1 ng/ml to 10 μg/mL, 5 ng/ml to 5 μg/mL, 10 ng/mL to 1 μg/mL, 20 ng/mL to 500 ng/mL, or 100 ng/ml to 500 ng/ml, and for example, 1.5 μg/mL. The medium may be supplemented with, in addition to laeverin, substances such as serum, a serum replacement, an antibiotic, an amino acid, 2-mercaptoethanol, and a growth factor. The medium may be serum-free. The culture period is not limited, and for example, is 1 to 30 days, 1 to 20 days, 1 to 14 days, or 1 to 10 days, and for example, 1, 2, 3, 4, 5, 6, 7, 8, 9, or 10 days.

The immune cell or the cell population containing immune cells may be cultured together with a laeverin-expressing cell(s) because the membrane-binding type laeverin is also effective. The term “laeverin-treated cell” used herein encompasses a cell cocultured together with a laeverin-expressing cell(s). The laeverin-expressing cell(s) can be a cell(s) into which a sequence encoding laeverin has been introduced, namely, a cell containing a transgene encoding laeverin. Such a laeverin-expressing cell(s) can be produced by introducing, into a cell, a vector containing a laeverin encoding sequence described herein. The cell into which the vector is to be introduced can be appropriately selected by those skilled in the art, and, for example, Swan71 cells can be used.

It was demonstrated that coculturing laeverin-expressing cells with PBMCs increases IDO1 expression in the PBMCs, suggesting that laeverin-expressing cells can evade immunity. Considering this, it is understood that expressing leaverin in cells by introducing a leaverin-encoding sequence into cells to be transplanted for cell transplantation therapy would increase the evasion of rejection in the host. Therefore, a laeverin-expressing cell containing a transgene encoding laeverin can be used in cell transplantation therapy. The transgene can encode any laeverin described herein. Examples of a cell into which a laeverin-encoding sequence is introduced include an iPS cell and a cell for transplantation induced from an iPS cell. This is particularly useful when an allogenic iPS cell is used in the therapy.

It was demonstrated that laeverin induces the expression of IFN-β, thereby inducing a group of ISGs, and induces the APOBEC3 family. Interferon is a cytokine produced by immune cells in the body during viral infection and possesses antiviral activity. IFN-β is clinically applied as a therapeutic agent for viral hepatitis, such as hepatitis B or hepatitis C, and is widely used. The APOBEC3 family is a cytidine deaminase known for inhibiting viral replication and conferring antiviral activity. Accordingly, laeverin can be used as an antiviral agent. Examples of target viruses include hepatitis B virus, hepatitis C virus, human immunodeficiency virus (HIV), and human papillomavirus (HPV). Examples of target viral diseases include hepatitis B, hepatitis C, HIV infection, and HPV infection. In addition, having cells for transplantation express laeverin confers, to the cells themselves, an effect of enhancing the secretion of IFN-β on immune cells, which means that transplantation cells resistant to an infectious disease or the like can be produced by having the cells express laeverina.

It was demonstrated that laeverin induces dendritic cells from monocytes. Accordingly, laeverin can be used for producing dendritic cells. A dendritic cell can be prepared by culturing a monocyte or a cell population containing monocytes in the presence of laeverin. Monocytes can be identified by CD14 expression and isolated from a monocyte-containing cell population. An example of the monocyte-containing cell population includes peripheral blood mononuclear cells. The peripheral blood mononuclear cells may be peripheral blood mononuclear cells collected from a subject to whom dendritic cells to be prepared is to be administered. Regarding medium, a medium typically used for culturing animal cells can be used, and for example, RPMI1640, MEM, IMDEM, DMEM, and the like can be used. Laeverin can be added to the medium at a final concentration of, for example, 1 ng/mL to 10 μg/mL, 5 ng/ml to 5 μg/mL, 10 ng/ml to 1 μg/mL, 20 ng/mL to 500 ng/mL, or 100 ng/ml to 500 ng/ml, and for example, 1.5 μg/mL. The medium may be supplemented with, in addition to laeverin, substances such as a serum, a serum replacement, an antibiotic, an amino acid, 2-mercaptoethanol, and a growth factor. The medium may be a serum-free medium. The culture period is not limited, and for example, is 1 to 30 days, 1 to 20 days, 1 to 14 days, or 1 to 10 days, and for example, 1, 2, 3, 4, 5, 6, 7, 8, 9, or 10 days. Whether dendritic cells are obtained can be determined by measuring a cell surface marker. Examples of the dendritic cell markers include CD11b, CD11c, CD123, CD38, CD80, and HLA-DR. The induction of a dendritic cell can be determined by measuring one or more selected markers. In one embodiment, a dendritic cell is a cell double-positive for CD11c/CD123. Accordingly, in one aspect, the present disclosure provides a method for preparing a dendritic cell, comprising culturing a cell population containing a monocyte in the presence of laeverin. Dendritic cells can be used in dendritic cell therapy for cancer, autoimmune disease, allergic disease, and the like.

A composition can comprise laeverin, a laeverin-treated cell(s), or a laeverin-expressing cell(s) as an active ingredient. The composition may comprise, in addition to laeverin or the cell, one or more pharmaceutically acceptable carriers. In the present disclosure, a pharmaceutically acceptable carrier means any substance, except for the active ingredient, that does not impair the effect of the active ingredient, and is highly safe in a subject. Examples of pharmaceutically acceptable carriers include excipients, stabilizers, buffers, preservatives, suspending agents, and tonicity agents. When the composition comprises the cell, the pharmaceutically acceptable carrier can, for example, be water, a medium, saline, an isotonic solution containing glucose or the like, or phosphate-buffered saline (PBS). The composition may include another active ingredient as long as the effect of laeverin or the cell is not impaired.

The composition can be in either a solid or a liquid form. Examples of dosage forms include, but are not limited to, capsules, tablets, powders, liquids, suspending agents, injections, ointments, creams, and suppositories. The composition can be prepared by a known method. In one embodiment, the composition is an injection. Examples of injections include aqueous injections, non-aqueous injections, suspension injections, emulsion injections, or solid injections that are to be dissolved or suspended in use.

The composition comprising laeverin, a laeverin-treated cell(s), or a laeverin-expressing cell(s) can be administered to a subject by a known administration method. Examples of administration methods include oral administration and parenteral administration. Examples of parenteral administration include intravenous, intrauterine, intramuscular, transdermal, and subcutaneous administration.

The subject may be a human or a non-human mammal. Examples of non-human mammals include rodents such as mice, rats, guinea pigs, and hamsters; non-human primates such as chimpanzees; artiodactyls such as cows, goats, and sheep; perissodactyls such as horses; and pet animals such as rabbits, dogs, and cats. In one embodiment, the subject is a human or a non-human primate (e.g., a chimpanzee, a monkey, a gorilla, a gibbon, or an orangutan). In another embodiment, the subject is a human.

The dose of laeverin or the cells is appropriately determined in accordance with factors such as the subject's age, weight, and condition, as well as the purpose of the treatment. When laeverin is administered to a subject, the dose of laeverin can be, for example, 1 mg, 5 mg, 10 mg, 15 mg, 20 mg, 25 mg, 30 mg, 35 mg, 40 mg, 45 mg, or 50 mg or more, and 10000 mg, 5000 g, 4500 mg, 4000 mg, 3500 mg, 3000 mg, 2500 mg, 2000 mg, 1500 mg, or 1000 mg or less per day for an adult. In one embodiment, the dose of laeverin is 15 mg to 4500 mg. When laeverin-treated cells or laeverin-expressing cells are administered to a subject, the number of cells to be administered can be, for example, 1 to 10¹⁰ cells, 10 to 10⁹ cells, 10² to 10⁸ cells, or 10³ to 10⁷ cells per day for an adult. The daily dose may be administered as a single dose or in multiple doses. The period of administration can be, but not limited to, one day or several days (e.g., 2, 3, 4, 5, or 6 days), one week or several weeks (e.g., 2, 3, 4, 5, or 6 weeks), one month or several months (e.g., 2, 3, 4, 5, or 6 months), or more. The administration can be performed every day, or once every few days (e.g., 2, 3, 4, 5, 6 days), every week or every few weeks (e.g., 2, 3, 4, 5, or 6 weeks), every month or every few months (e.g., 2, 3, 4, 5, or 6 months).

It was demonstrated that the expression of ISG15 and IDO1 by stimulation with laeverin and the ratio in expression level between ISG15 and IDO1 (referred to as an IDO1/ISG15 ratio) in peripheral blood mononuclear cells are lower in women having a hypertensive disorder of pregnancy than in normal pregnant women, and it was suggested that these values are involved in these diseases. In addition, it was demonstrated that the proportion of laeverin-positive T cells is high in women having a hypertensive disorder of pregnancy or systemic lupus erythematosus (SLE), and it was suggested that laeverin-positive T cells are involved in these diseases. Therefore, based on the ISG15 expression level, the IDO1 expression level, or the IDO1/ISG15 ratio in the peripheral blood mononuclear cells of a subject, or based on the amount or proportion of laeverin-positive T cells, a disease or onset risk of the disease can be determined, or the therapeutic effect of a therapeutic agent can be evaluated. Examples of diseases include hypertensive disorders of pregnancy and immune-related diseases such as SLE.

In one embodiment of the method of the present disclosure, peripheral blood mononuclear cells collected from a subject are cultured in the presence of laeverin, and thereafter, the ISG15 expression level, the IDO1 expression level, or the IDO1/ISG15 ratio in the peripheral blood mononuclear cells is compared with a corresponding reference value. Regarding the medium, any commonly used medium for culturing animal cells can be used; for example, RPMI 1640, MEM, IMDM, DMEM, and the like. Laeverin can be added to a medium at a final concentration of, for example, 1 ng/ml to 10 μg/mL, 5 ng/ml to 5 μg/mL, 10 ng/ml to 1 μg/mL, 20 ng/mL to 500 ng/ml, or 100 ng/mL to 500 ng/mL, and for example, 1.5 μg/mL. The medium may be supplemented with, in addition to laeverin, substances such as serum, a serum replacement, an antibiotic, an amino acid, 2-mercaptoethanol, and a growth factor. The medium may be serum-free. Culturing time is not limited; it can be, for example, 4 to 24 hours, 6 to 24 hours, 8 to 24 hours, 10 to 24 hours, or 12 to 24 hours. Collecting peripheral blood mononuclear cells from a subject and measuring ISG15 or IDO1 expression level can be performed by a general method. For example, the ISG15 and IDO1 expression levels can be measured by PCR (polymerase chain reaction), microarray, or the like. The method of the present disclosure may comprise comparing two or more selected from the ISG15 expression level, the IDO1 expression level, and the IDO1/ISG15 ratio, respectively, with corresponding reference values.

In one embodiment, the method of the present disclosure comprises comparing the amount or proportion of laeverin-positive T cells in peripheral blood collected from a subject to a reference value. The proportion of laeverin-positive T cells in peripheral blood means the proportion of laeverin-positive T cells in peripheral blood mononuclear cells contained in the peripheral blood. The collection of peripheral blood and peripheral blood mononuclear cells from a subject, and the measurement of laeverin-positive T cells, can be performed by a general method. For example, laeverin-positive T cells in peripheral blood can be measured by flow cytometry, using an anti-CD3 antibody and an anti-laeverin antibody to stain peripheral blood mononuclear cells.

The reference value can be a value of the IGS15 expression level, the IDO1 expression level, the IDO1/ISG15 ratio, or the amount or proportion of laeverin positive T cells with which a group of subjects confirmed to have the disease can be distinguished from a group of subjects not having the disease (e.g., a group of healthy persons) with a statistically significant difference. The statistically significant difference can be analyzed using methods such as the chi-square test, the generalized Wilcoxon test, the Wilcoxon signed-rank test, the Mann-Whitney test, the log-rank test, or the Cox proportional hazards model. The reference value may be set based on sensitivity and/or specificity. Sensitivity is the true-positive rate, and specificity is the true-negative rate. For example, a value corresponding to a high positive rate in the group of subjects confirmed to have the disease or a high negative rate in the group of subjects not having the disease may be defined as the reference value.

In one embodiment, when the ISG15 expression level, the IDO1 expression level, or the IDO1/ISG15 ratio in the peripheral blood mononuclear cells collected from a subject and cultured in the presence of laeverin is lower than the reference value, or when the amount or proportion of laeverin-positive T cells in the peripheral blood collected from a subject is higher than the reference value, it is determined that the subject has the disease, or has a risk of onset of the disease.

When the effect of a therapeutic agent against a disease is to be evaluated, the peripheral blood or peripheral blood mononuclear cells are collected from a subject having the disease after administration of the therapeutic agent. In one embodiment, when the ISG15 expression level, the IDO1 expression level, or the IDO1/ISG15 ratio in the peripheral blood mononuclear cells collected from the subject having the disease after administration of the therapeutic agent is higher than the reference value, or the amount or proportion of laeverin-positive T cells in the peripheral blood collected from the subject having the disease after administration of the therapeutic agent is lower than the reference value, it is determined that the therapeutic agent is effective. In this case, the reference value may be a reference value defined as described above, or alternatively, may be the ISG15 expression level, the IDO1 expression level, or the IDO1/ISG15 ratio in the peripheral blood mononuclear cells collected from the same subject before the administration of the therapeutic agent, or the amount or proportion of laeverin-positive T cells in the peripheral blood collected from the same subject before the administration of the therapeutic agent.

Exemplary embodiments of the present disclosure are as follows:

[1]

A composition for inducing immunotolerance, comprising laeverin.

[2]

The composition according to item 1, wherein the composition is administered to an autoimmune disease patient.

[3]

The composition according to item 1, wherein the composition is administered to a recipient of organ transplantation.

[4]

The composition according to item 1, wherein the composition is administered into the uterus prior to embryo transfer after in-vitro fertilization.

[5]

The composition according to item 1, wherein the composition is used in in-vitro treatment of an immune cell or a cell population containing immune cells.

[6]

The composition according to item 5, wherein the immune cell or the cell population containing the immune cells is a cell(s) collected from an autoimmune disease patient.

[7]

The composition according to item 5, wherein the immune cell or the cell population containing the immune cells is a cell(s) collected from a recipient of organ transplantation.

[8]

The composition according to item 5, wherein the immune cell or the cell population containing the immune cells is a cell(s) collected from a recipient of embryo transfer.

[9]

The composition according to any one of items 1 to 8, wherein the laeverin is a polypeptide comprising or consisting of an amino acid sequence having 80% or more, 85% or more, 90% or more, 95% or more, 96% or more, 97% or more, 98% or more, or 99% or more sequence identity to the amino acid sequence shown in SEQ ID NO: 1.

[10]

The composition according to item 9, wherein the laeverin is a polypeptide comprising or consisting of the amino acid sequence shown in SEQ ID NO: 1.

[11]

The composition according to any one of items 1 to 8, wherein the laeverin is a polypeptide comprising or consisting of an amino acid sequence having 80% or more, 85% or more, 90% or more, 95% or more, 96% or more, 97% or more, 98% or more, or 99% or more sequence identity to the amino acid sequence consisting of 65th to 990th amino acids shown in SEQ ID NO: 1.

[12]

The composition according to item 11, wherein the laeverin is a polypeptide comprising or consisting of the amino acid sequence consisting of 65th to 990th amino acids shown in SEQ ID NO: 1.

[13]

A method for inducing immunotolerance, comprising administering an effective amount of laeverin to a subject in need thereof.

[14]

The method according to item 13, wherein the subject is an autoimmune disease patient.

[15]

The method according to item 13, wherein the subject is a recipient of organ transplantation.

[16]

The method according to item 13, wherein the subject is a recipient of embryo transfer.

[17]

A method for treating a cell, comprising culturing an immune cell or a cell population containing immune cells in the presence of laeverin.

[18]

The method according to item 17, wherein the cell population consists of peripheral blood mononuclear cells.

[19]

A composition for cell transplantation therapy, comprising a laeverin-expressing cell comprising a transgene encoding laeverin.

[20]

An antiviral agent, comprising laeverin.

[21]

A method for treating a viral disease, comprising administering an effective amount of laeverin to a subject in need thereof.

[22]

A composition for inducing a dendritic cell, comprising laeverin.

[23]

A method for preparing a dendritic cell, comprising culturing a monocyte or a cell population containing monocytes in the presence of laeverin.

[24]

The method according to item 23, wherein the cell population consists of peripheral blood mononuclear cells.

[25]

A method for determining a disease or a risk of onset of the disease, comprising:
culturing, in the presence of laeverin, peripheral blood mononuclear cells collected from a subject; and
comparing, to a reference value, ISG15 expression level, IDO1 expression level, or an IDO1/ISG15 ratio in the peripheral blood mononuclear cells after the culturing.
   [26]

A method for evaluating an effect of a therapeutic agent, comprising:
culturing, in the presence of laeverin, peripheral blood mononuclear cells collected from a subject with a disease after administration of the therapeutic agent; and
comparing, to a reference value, ISG15 expression level, IDO1 expression level, or an IDO1/ISG15 ratio in the peripheral blood mononuclear cells after the culturing.
   [27]

The method according to item 25 or 26, wherein the disease is a hypertensive disorder of pregnancy.

[28]

A method for determining a disease or a risk of onset of the disease, comprising comparing, to a reference value, an amount or proportion of laeverin-positive T cells in peripheral blood collected from a subject.

[29]

A method for evaluating an effect of a therapeutic agent, comprising comparing, to a reference value, an amount or proportion of laeverin-positive T cells in peripheral blood collected from a subject with a disease after administration of the therapeutic agent.

[30]

The method according to item 28 or 29, wherein the disease is a hypertensive disorder of pregnancy.

The present invention will be further described below by way of examples, and it is noted that the present invention is not limited by these examples in any sense.

### EXAMPLES

### I. Methods

1. Production of Recombinant Laeverin (rLVRN)

A cDNA encoding full-length human laeverin (LVRN) having a hexahistidine (His6) tag at its C terminus (1 to 990) was produced by PCR. The amplified cDNA was cloned into the EcoRI-XhoI site of a baculoviral transfer vector, pFastBac1 vector (Invitrogen). Next, the prepared transfer vector was introduced into E. coli DH10Bac cells harboring a baculoviral genome (bacmid) and a transposition helper vector (Invitrogen), and thus, a recombinant bacmid DNA containing LVRN cDNA was produced. Subsequently, the bacmid DNA was introduced into Sf9 insect cells using a Collection (R) Reagent (Invitrogen). The cells were cultured for 72 hours, and the recombinant baculovirus was collected. To express rLVRN, the Sf9 cells (2.0×10⁶/ml) infected with the recombinant baculovirus (infection rate: about 1 to 3) were cultured in 3 liters of SFM-900III medium (Invitrogen) supplied with 8.0 ppm of O₂ (Cellmaster-1700, WAKENYAKU CO., LTD., Kyoto, Japan) at 27° C. for 72 hours. The medium containing soluble rLVRN was collected by centrifugation, applied to a hydroxyapatite column (2.5×10 cm, Nacalai Tesque, Kyoto, Japan) equilibrated with 50 mM Tris/HCl buffer (pH 7.5), and eluted with 100 mM sodium phosphate buffer (pH 7.5). The resultant eluate was applied to a chelating sepharose column (1.0×10 cm) (GE Healthcare Bio-Science, Piscataway, NJ) previously loaded with Ni²⁺ and eluted with 200 mM imidazole. The secretory-type LVRN with the His6 tag at the C terminus (LVRN (65 to 990)+His6) was obtained.

2. Production of Enzymatic Activity-Inactivated rLVRN

An enzymatic activity-inactivated rLVRN (E416Q rLVRN) was made by site-directed mutagenesis. A cDNA encoding E416Q rLVRN was produced with QuikChange Site-Directed Mutagenesis Kit (Stratagene, LaJolla, CA). To express it as a soluble protein, the sequence encoding a cytoplasm region and a transmembrane region of the enzyme (Met1 to Gln64) was replaced with a signal peptide (MNLLLILTFVAAVAA) (SEQ ID NO: 3) of human trypsin II, and a His6 tag was added to the C terminus. The amplified cDNA was cloned into the BssHII-XhoI site of a baculoviral transfer vector, pFastBac-1 (Invitrogen). To produce a recombinant bacmid DNA containing the cDNA of E416Q rLVRN, the produced transfer vector was transformed into E. coli DH10Bac cells harboring a baculoviral genome (bacmid) and a transposition helper vector (Invitrogen). The bacmid DNA was introduced into Sf9 insect cells using a Cellfectin (R) Reagent (Invitrogen), and the resultant cells were cultured for 72 hours to collect the recombinant baculovirus. To express E416Q rLVRN, the Sf9 cells (2.0×10⁶/ml) infected with the recombinant baculovirus (infection rate: about 1 to 3) were cultured in 3 liters of SFM-900III medium (Invitrogen) supplied with 8.0 ppm of O₂ (Cellmaster-1700, WAKENYAKU CO., LTD., Kyoto, Japan) at 27° C. for 72 hours. The medium containing E416Q rLVRN was collected by centrifugation, applied to a hydroxyapatite column (2.5×10 cm) (Nacalai Tesque, Kyoto, Japan) equilibrated with 50 mM Tris/HCl buffer (pH 7.5), and eluted with 100 mM sodium phosphate buffer (pH 7.5). The resultant eluate was applied to a Ni²⁺ chelating sepharose column (1.0×10 cm) (GE Healthcare Bio-Science, Piscataway, NJ), and eluted with 200 mM imidazole.

### 3. Microarray Analysis and Gene Ontology Analysis

Gene expression array analysis using a microarray was performed according to the manufacturer's instructions. Specifically, a Low Input Quick Amp Labeling Kit (Agilent Technologies, Sant Clara, CA, USA) was used to prepare a labeling cDNA from 0.2 μg of RNA. A sample was hybridized to a SurePrint G3 Human GE microarray 8×60 K Ver. 3.0 (G4845A #72363, Agilent Technologies), and the resultant was scanned with Agilent DNA Microarray Scanner (G2539A) using one-color scan setting of 1×60 k array sides (Agilent Technologies). The scanned image was analyzed with Feature Extraction Software 11.0.1.1 (Agilent). Data were normalized with GeneSpring software 12.1 (Agilent Technologies), and filtered with 3 filters. From normalized intensity data of the microarray, differentially expressed genes were determined by the weighted average difference (WAD) ranking method (Kadota K, Nakai Y, Shimizu K., A weighted average difference method for detecting differentially expressed genes from microarray data. Algorithms Mol Biol. 2008 Jun. 26; 3:8. doi: 10.1186/1748-7188-3-8. PMID: 18578891; PMCID: PMC2464587). This method is a statistical approach based not only on differences in gene expression but also on fold-change analysis of microarray signal intensities. The identified genes whose expression increased or decreased were subjected to gene ontology analysis using the Database for Annotation, Visualization and Integrated Discovery (DAVID), with a P value <0.05 used as the threshold.

### 4. Collection and Separation of Peripheral Blood Mononuclear Cell (PBMC)

Blood was collected, with heparin addition, from healthy men, non-pregnant women, normal pregnant women, and pregnant women with a complication. PMBCs were isolated from the collected blood with a Ficoll-Paque™ PLUS (cytiva, density 1.077±0.01 g/mL). The isolated PBMCs were cryopreserved with CELLBANKER1 in a liquid nitrogen tank. In experiments, the PMBCs from healthy non-pregnant women were used unless otherwise stated.

### 5. Quantitative PCR

Total RNA was extracted from the cells using an RNeasy Mini Kit (Qiagen, Hilden, Germany) according to the manufacturer's instructions. The RNA was reverse-transcribed into cDNA using the PrimeScript™ RT-PCR Kit (Takara Bio Inc., Shiga, Japan). As a control, β-actin (ACTB) was used. The cDNA was amplified by using forward (Fd) and reverse (Rs) primers specific to each gene (Table 1). PCR was conducted using a 2-step PCR protocol. The cycling conditions included an initial denaturation at 95° C. for 30 seconds, 40 to 45 cycles of denaturation at 95° C. for 5 seconds and annealing/extension reaction at 60° C. for 30 seconds. Quantitative real-time PCR analysis was conducted using AriaMX Real-Time PCR System (Agilent Stratagene, LaJolla, CA, USA) according to the manufacturer's instructions. Averages±SD of the results of 3 to 5 independent experiments are shown.

TABLE 1

| | | SEQ | | SEQ | |
|---|---|---|---|---|---|
| | | ID | | ID | |
| gene | Forward 5→3 | NO. | Reverse 3→5 | NO. | Length |
| IFIT1 | TCTCAGAGGAGCCTGGCTAA | 4 | CCAGACTATCCTTGACCTGATGA | 5 | 102 |
| IFIT3 | CAGAACTGCAGGGAAACAGC | 6 | TGAATAAGTTCCAGGTGAAATGGC | 7 | 100 |
| ISG15 | GAGAGGCAGCGAACTCATCT | 8 | CTTCAGCTCTGACACCGAGA | 9 | 157 |
| OAS2 | AGGTGGCTCCTATGGACGGAA | 10 | GGCTTCTCTTCTGATCCTGGAATTG | 11 | 104 |
| ACTB | TGGCACCCAGCACAATGAA | 12 | CTAAGTCATAGTCCGCCTAGAAGCA | 13 | 186 |
| IDO1 | GGCACACGCTATGGAAAACT | 14 | CGGACATCTCCATGACCTTT | 15 | 297 |
| IFN α | TGGGCTGTGATCTGCCTCAAAC | 16 | CAGCCTTTTGGAACTGGTTGCC | 17 | 156 |
| IFN β | ACCATCTGAAGACAGTCCTGG | 18 | CCTTGGCCTTCAGGTAATGC | 19 | 123 |
| IFN γ | GAGTGTGGAGACCATCAAGGAAG | 20 | TGCTTTGCGTTGGACATTCAAGTC | 21 | 124 |
| LAG3 | GCAGTGTACTTCACAGAGCTGTC | 22 | AAGCCAAAGGCTCCAGTCACCA | 23 | 143 |
| CD14 | CTGGAACAGGTGCCTAAAGGAC | 24 | GTCCAGTGTCAGGTTATCCACC | 25 | 120 |
| CD1Ic | CAGTCATCACAGCGGTACTGTACA | 26 | TCCATTTGCCTCCTCCATC | 27 | 77 |
| CD123 | TTAAGCAGGCACCTCTGTCC | 28 | CTGAGCCTTTGCTTTCATCC | 29 | 154 |
| CD80 | CTCTTGGTGCTGGCTGGTCTTT | 30 | GCCAGTAGATGCGAGTTTGTGC | 31 | 136 |
| CD86 | CCATCAGCTTGTCTGTTTCATTCC | 32 | GCTGTAATCCAAGGAATGTGGTC | 33 | 154 |
| PDL1 | AAACAATTAGACCTGGCTG | 34 | TCTTACCACTCAGGACTTG | 35 | 400 |

### 6. Flowcytometry

PBMCs or Swan71 cells were incubated at 4° C. for 30 minutes with an FITC-binding anti-LVRN antibody (clone 5-23) or an FITC-binding mouse IgG antibody (clone: P3.6.2.8.1, Lot: 2067148, Invitrogen) as a control. Cell-surface labeling was analyzed by fluorescence detection using a BD FACS Aria™ Fusion cell sorter.

FACS analysis of ISG15 expression in immune cells in the presence of rLVRN was conducted as follows. After culturing PBMCs with 1.5 μg/mL of rLVRN added thereto for 24 hours, the PBMCs were collected, and then treated with a BD Pharmingen™ Human BD Fc block™ (Cat #564220, LOT: 9049805) at 4° C. for 30 minutes for blocking. An FITC-binding CD14 (monocyte marker) antibody (FITC mouse IgG1k anti-human CD14 Clone: HCD14, Cat #325604, LOT: B268830, Biolegend), an APC-binding anti-CD19 (B cell marker) antibody (APC mouse IgG1k anti-human CD19, Cat #555415, LOT: 8296813, BD pharmingen), and a BV42-binding CD3 (T cell marker) antibody (BV421 mouse IgGlk anti-human CD3 Clone: UCHT1 Cat #562426, LOT: 9113553) were allowed to act on the resultant at 4° C. for 1 hour. Subsequently, the antibodies were washed several times, then the resultant was immobilized with 1% PFA for 10 minutes, and subjected to membrane permeabilization treatment with 0.1% Triton/PBS for 10 minutes. Ultimately, a PE-binding anti-ISG15 antibody (anti-hISG15/UCRP PE PE-conjugated Rat IgG2A, Cat #IC8044P, LOT: ADRU0219021, R & D Systems) was reacted thereon at 45° C. for 1 hour to perform analysis by fluorescence detection with an ACS Aria™ Fusion cell sorter.

Analysis of LVRN-expressing positive T cells in maternal blood was conducted as follows. PBMCs isolated from collected blood were treated with a BD Pharmingen™ Human BD Fc block™ (Cat #564220, LOT: 9049805) at 4° C. for 30 minutes for blocking. After washing the resultant several times, an FITC-binding anti-LVRN antibody (clone 5-23) and a BV421-binding CD3 (T cell marker) antibody (BV421 mouse IgG1k anti-human CD3 Clone: UCHT1, Cat #562426, LOT: 9113553) were allowed to act thereon at 4° C. for 1 hour for double staining. Subsequently, after washing the antibodies several times, the resultant was analyzed by fluorescence detection with BD FACS Aria™ Fusion cell sorter.

Analysis of differentiation of CD14 cells using rLVRN was analyzed as follows. After culturing PBMCs with 1.5 μg/mL of rLVRN added thereto for 24 hours, the resultant PBMCs were collected and treated with BD Pharmingen™ Human BD Fc block™ (Cat #564220, LOT: 9049805) at 4° C. for 30 minutes for blocking. After immobilizing the resultant with 1% PFA for 10 minutes, an FITC-binding CD14 (monocyte marker) antibody (FITC mouse IgG1k anti-human CD14 Clone: HCD14, Cat #325604, LOT: B268830, Biolegend), a PE-binding anti-HLA-DR (antigen presenting cell marker) antibody (PE mouse IgG2ak anti-human HLA-DR, Cat #555812, LOT: 9248092, BD Pharmingen), an APC-binding anti-CD11c (dendritic cell marker (mainly moDC)) antibody (APC mouse IgG1k anti-human CD11c Clone: 3.9, Cat #301614, LOT: B276681, Biolegend), and a BV421-binding anti-CD123 (dendritic cell marker (mainly pDC)) antibody (BV421 mouse IgG2ak anti-human CD123 Clone: 7G3, Cat #563362, LOT: 0279696, BD Horizon) were allowed to act on the resultant at 4° C. for 1 hour, which was analyzed by fluorescence detection with a BD FACS Aria™ Fusion cell sorter.

7. Production of Fluorescently Labeled rLVRN, and Analysis of Binding and Cell Incorporation of Fluorescently Labeled rLVRN

rLVRN was fluorescently labeled with the AnaTag HyLite Fluor 488 Microscale Protein Labelling Kit (AnaSpec Inc., Fremont, CA, USA) according to the manual. After culturing PBMCs with 1.5 μg/mL of the fluorescently labeled rLVRN added thereto for 4 hours or 24 hours, the resultant PBMCs were collected, immobilized with 4% PFA for 10 minutes, and separated into a monocyte fraction and a lymphocyte fraction with the BD FACS Aria™ Fusion cell sorter to analyze fluorescent intensities thereof.

8. Absorption Experiment of rLVRN with Beads

An experiment of suppression by rLVRN on beads capable of binding the histidine tag of the rLVRN was conducted. To 400 μL of PBS, 1 μL of Bc Mag™ His-Ni magnetic Beads (Cat #MHN-102, LOT: 0119), and 4.5 μg of rLVRN were added, followed by stirring at 4° C. After 30 minutes, a magnet (6-Tube Magnetic Separation Rack #7017S, LOT: 0001, Cell Signaling) was used to collect the beads and rLVRN having reacted with the beads, and only a supernatant was added to PBMCs. The resultant was incubated at 37° C. for 24 hours, and the PBMCs were collected for RNA extraction.

### 9. Establishment of LVRN-Swan71 Cells

For establishing Swan71 cell line expressing LVRN, a cDNA of full-length human laeverin with His6 tag at the C terminus (hLVRN-His) was inserted into pTargeT vector (Promega), the resulting rLVRN-His/pTargeT (500 ng) was linearized through cleavage with Psp 1406I, and was introduced into Swan71 cells with FuGeneHD (Promega). 48 hours after gene transfer, a medium supplemented with 500 g/mL G418 was used to establish a stably expressing cell line, which was isolated. To establish NEO-Swan71 cells, pTargeT vector was used instead of rLVRN-His/p TargeT.

10. Direct Culturing with LVRN-Swan71 Cell

LVRN-Swan71 cells and NEO-Swan71 cells, previously treated with 2.5 g/mL of mitomycin for 4 hours, were cultured in a 12-well adherent cell plate at a concentration of 1.5×10⁵ cells/well under the condition of 1 mL/well of RPMI. After checking cell adhesion, PBMCs were adjusted to 1.5×10⁵ cells/mL in RPMI, and the resulting 1 mL was substituted for the culture medium of LVRN-Swan71 cells and NEO-Swan71 cells. The resultants were incubated at 37° C. for 24 hours, and then, the culture plate was stirred on a shaker for 10 minutes. The cells were collected from the supernatant and filtered twice through a cell strainer with a 35 μm pore size. The resultant was further reacted with CD45 MicroBeads (Order No: 130-045-801, LOT: 5151027008, Miltenyi Biotec), and CD45-positive cells were selectively isolated and collected using the Possel program of autoMACS® Pro Separator (Miltenyi Biotec).

11. Indirect Culturing with LVRN-Swan71 Cell

LVRN-Swan71 cells and NEO-Swan71 cells, previously treated with 2 g/mL of mitomycin for 4 hours, were adjusted with RPMI to 3.0×10⁵ cells/well in 3 mL/well of a 6-well adherent cell plate and cultured. After checking cell adhesion, a 0.4 μm transwell (Greiner Bio-One (R)) holding PBMCs (1.0×10⁵ cells/well) was placed thereon, incubated at 37° C. for 24 hours, and the PBMCs in the transwell were collected.

12. Inhibition Experiment with IFN-Neutralizing Antibody and Type-I IFN Receptor Neutralizing Antibody

PBMCs were adjusted with RPMI to a concentration of 7×10⁵ cells/mL in a 12-well suspension cell culture plate and the resultant 1 ml was cultured. Thereto added were 1 μg/mL of a type-I IFN receptor neutralizing antibody (Interferon alpha/beta Receptor Chain 2 Cat #MAB1155, LOT: 3766724 EMD, Millipore corporation) and 1 μg/mL of an IFN-β neutralizing antibody (anti-hIFN-β, AF814, LOT: BTT0818121). After the incubation at 37° C. for 10 minutes, 1.5 μg/mL of rLVRN or 100 IU/mL of IFNβ (Interferon Beta, Daiichi Sankyo Co., Ltd.) was added thereto. After further incubation at 37° C. for 24 hours, the cells were collected.

13. Inhibition Experiment with JAK1,2 Inhibitor Ruxolitinib

PBMCs were adjusted to a concentration of 1.5×10⁵ cells/mL with RPMI, the resultant 1 mL was cultured in a 12 well suspension cell culture plate. 1 μM of JAK inhibitor, ruxolitinib (Ruxolitinib INCB18424, Cat #S1378, LOT: S137815, Selleckchem), was added thereto, and the resultant was incubated at 37° C. for 10 minutes. Thereafter, 1.5 μg/mL of rLVRN was added thereto, and the resultant was further incubated at 37° C. for 24 hours, and then, the resultant cells were collected for extracting RNA.

### 14. Method for Separating CD14-Positive Monocyte Fraction, CD4-Positive Fraction, or CD8-Positive Fraction

For separating/collecting CD14-positive monocytes, CD14 microbeads (CD14 MicroBeads, human, Order no: 130-050-201, Lot no: 5210205621, Miltenyi Biotec) were reacted, and CD45-positive cells were selectively isolated and collected by using the Possel program of autoMACS® Pro Separator (Miltenyi Biotec). Separation and collection of CD4-positive cells and CD8-positive cells were conducted in the same manner by using CD4 microbeads (CD4 MicroBeads, human, Order no: 130-045-101, Lot no: 5210401362, Miltenyi Biotec), or CD8 microbeads (CD8 MicroBeads, human, Order no: 130-045-201, Lot no: 5210401341, Miltenyi Biotec).

### 15. Cell Staining

For staining Swan71 cells, after the cells were immobilized with 4% PFA for 10 minutes, and an anti-LVRN antibody clone (5-23) was allowed to act thereon, Alexa Flour 488 goat anti-mouse IgG (H+L) (REF: A11001, LOT: 2220848, Invitrogen) was allowed to act thereon. Then, the resultant was embedded and observed with a fluorescence microscope (Olympus BX50 microscope) and photographed with a camera (DP72 Olympus digital camera).

Expression of ISG15 by rLVRN was observed in immune cells as follows. PBMCs were supplemented with rLVRN (1.5 μg/mL) and cultured for 24 hours. Then, the PBMCs were collected and immobilized with 4% PFA for 10 minutes, then subjected to membrane permeability treatment with 0.1% triton/PBS for 10 minutes, and blocked with 1% FBS/PBS for 30 minutes. An anti-CD14 (monocyte marker) antibody (CD14 (UCH-M1) mouse mAb sc-1182, LOT: K1308, Santa Cruz), an anti-CD19 (B cell marker) antibody (CD19 (EPR5906) rabbit Ab ab134114, LOT: GR3252252-1), an anti-CD4 antibody (CD4 (BC/1F6) mouse mAb ab846, LOT: GR235014-1), an anti-CD8 antibody (CD8 Clone C8/144B mouse mAb anti-human, REF: M7103, LOT: 20024879, DAKO), and a PE-binding anti-ISG15 antibody (anti-hISG15/UCRP PE conjugated Rat IgG2A, Cat #IC8044P, LOT: ADRU0219021, R & D systems) were allowed to act thereto at room temperature for 2 hours. Subsequently, after the washing of the antibodies several times, Alexa Fluor 488 goat anti-rabbit IgG (H+L) (REF: A11034, LOT: 2286890, Invitrogen) or Alexa Fluor 488 goat anti-mouse IgG (H+L) (REF: A11001, LOT: 2220848, Invitrogen) was allowed to act as a secondary antibody on the resultant at room temperature for 1 hour. After washing several times, the resultant was ultimately subjected to nucleic acid staining with 1 μg/mL of Hoechst (R) 33342. After washing the antibodies several times, centrifuged cells were suspended in 20 μL of PBS, 20 μL of the suspension was dropped onto a glass slide, covered with a cover glass, embedded, observed with a fluorescence microscope (Olympus BX50 microscope) and photographed with a camera (DP72 Olympus digital camera).

The incorporation of rLVRN by CD14 cells was checked as follows. PBMCs were cultured with HiLyte Fluor™ 555 dye-labeled rLVRN (1.5 μg/mL) for 24 hours and then were collected, and treated with BD Pharmingen™ Human BD Fc block™ (Cat #564220, LOT: 9049805) at 4° C. for 30 minutes for blocking. The resultant was fixed with 1% PFA for 10 minutes, and an anti-CD14 antibody (CD14 (UCH-M1) mouse mAb sc-1182, LOT: K1308, Santa Cruz) was applied at 4° C. for 1 hour. Subsequently, the antibody was washed several times, and Alexa Fluor 488 goat anti-mouse IgG (H+L) (REF: A11001, LOT: 2220848, Invitrogen) as a secondary antibody and Hoechst (R) 33342 for staining nucleic acids were allowed to act thereon at room temperature for 2 hours. After washing several times, centrifuged cells were suspended in 200 μL of PBS, 50 μL of the suspension was dropped onto a glass slide, covered with a cover glass, embedded, observed with a fluorescence microscope (Olympus BX50 microscope), and photographed with a camera (DP72 Olympus digital camera).

### 16. Activation of THP-1 Cell

The human acute monocytic leukemia cell line THP-1 (RRID: CVCL_0006), which exhibits monocyte-like characteristics, was purchased from ATCC. THP-1 cells were cultured in RPMI (Nacalai Tesque, Kyoto, Japan) supplemented with 10% FBS (Sigma-Aldrich), 100 g/mL streptomycin, and 100 IU/mL penicillin at 5% CO₂ and 37° C. To activate the THP-1 cells, PMA (phorbol 12-myristate 13-acetate) (Sigma-Aldrich) was added, followed by culturing for 48 hours.

### 17. Western Blotting Analysis

Expression of IDO1 protein and ACTB protein in THP-1 cells activated by the treatment with PBS or rLVRN (1.5 or 7.5 μg/mL) for 12 hours or 24 hours was measured by Western blotting analysis. The Western blotting analysis was conducted as described in the literature (Matsumoto, T., Iizuka, T., Nakamura, M., Suzuki, T., Yamamoto, M., Ono, M., Kagami, K., Kasama, H., Wakae, K., Muramatsu, M., et al. (2022). FOXP4 inhibits squamous differentiation of atypical cells in cervical intraepithelial neoplasia via an ELF3-dependent pathway. Cancer Sci. 10.1111/cas. 15489.). A protein lysate was extracted with RIPA Buffer (Cell Signaling Technology Inc., Danvers, MA, USA), then electrophoresed on a 10% SDS-PAGE gel and transferred to a nitrocellulose membrane. The transferred membrane was incubated overnight at 4° C. together with a primary antibody against IDO (1:1000, Cell Signaling Technology, Cat #86630, RRID: AB_2636818) or Actin (1:5,000, Santa Cruz Biotechnology, Cat #sc-1615, RRID: AB_630835). A horseradish peroxidase (HRP)-binding antibody was applied as a secondary antibody at room temperature for 1 hour. The blot was visualized with an enhanced chemiluminescence system using ECL™ Western Blotting Detection Reagents (GE Healthcare, Buckinghamshire). For IDO1 protein level, using ImageJ, the IDO1 and ACTB bands were quantified, and the density ratio was calculated and normalized to the ACACTB protein. In a part of the experiment, the IDO1 protein in Hela cells treated with 10 ng/ml of IFN-5 for 24 hours was used as a positive control.

### 18. High Performance Liquid Chromatography-Mass Spectrometry (HPLC-MS)

The levels of tryptophan and kynurenine in the culture fluid were quantitatively determined by high performance liquid chromatography, a high-resolution mass spectrometer, and a tandem mass spectrometer (HPLC-MS/MS). In order to evaluate a difference between PBS treatment and rLVRN treatment, PMA activated THP-1 cells were treated with 1.5 or 7.5 μg/mL of r-LVRN for 48 hours. Thereafter, the amount of metabolites in the culture fluid was measured in Institute of Genetics, Kanazawa University by HPLC-MS as described in literature (Dochi, H., Kondo, S., Murata, T., Fukuyo, M., Nanbo, A., Wakae, K., Jiang, W. P., Hamabe-Horiike, T., Tanaka, M., Nishiuchi, T., et al. (2022). Estrogen induces the expression of EBV lytic protein ZEBRA, a marker of poor prognosis in nasopharyngeal carcinoma. Cancer Sci 113, 2862-2877. 10.1111/cas.15440.). A kynurenine/tryptophan ratio was calculated to evaluate the activity of IDO1.

### II. Results

### 1. Induction of ISGs, IDO1, and PD-L1 Genes in PBMC by Laeverin

### 1A: Gene Ontology Analysis by Microarray

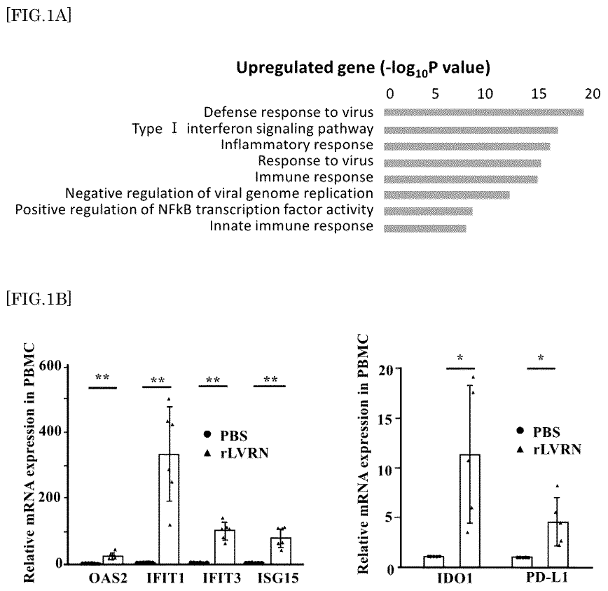

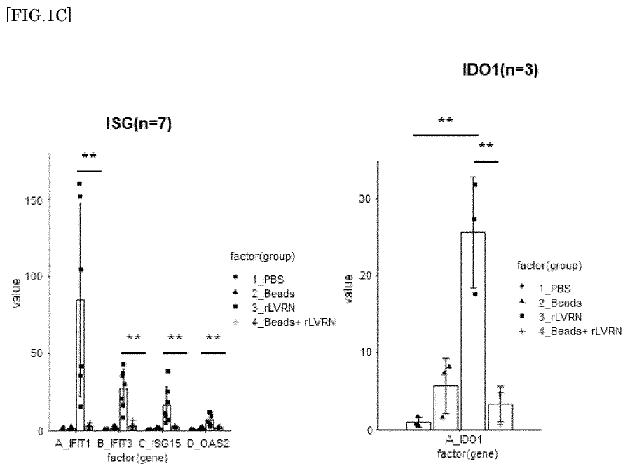

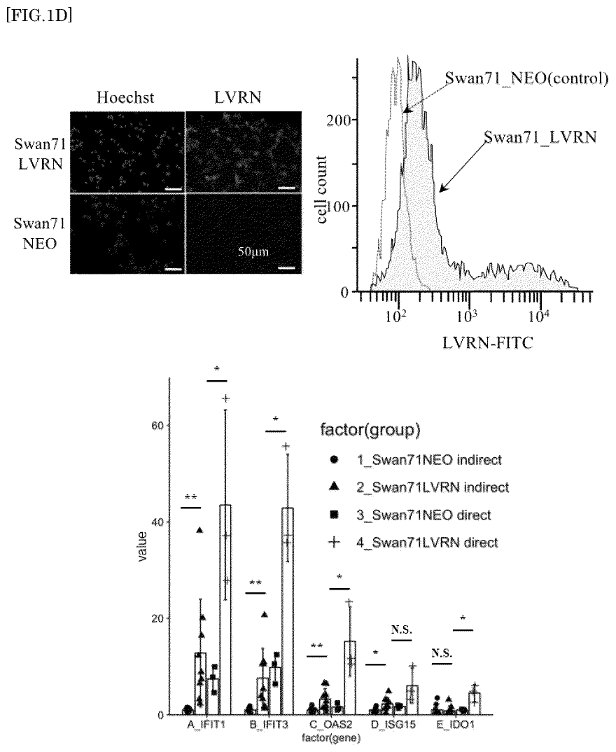

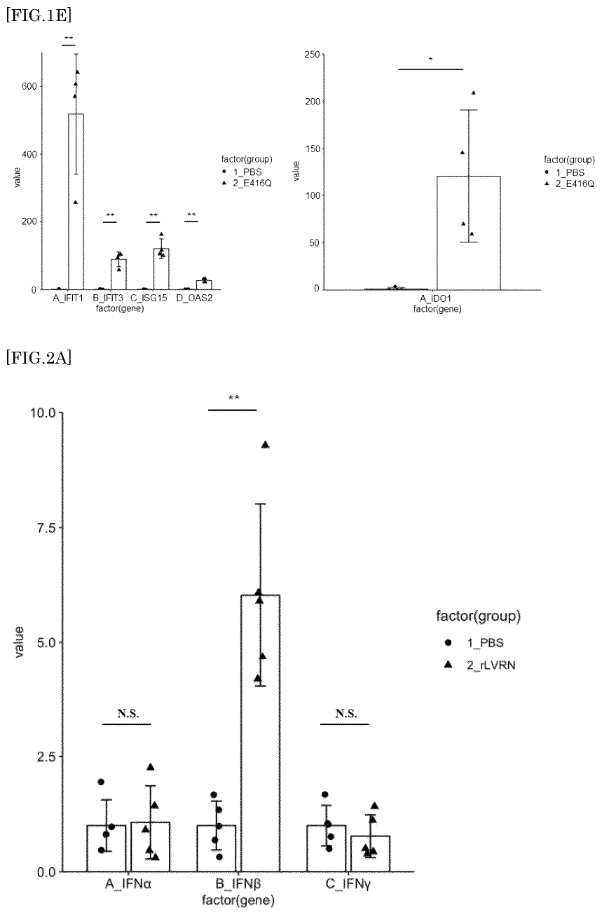

PBMCs were supplemented with rLVRN (1.5 μg/mL) and cultured for 24 hours, and a group of genes changed in the expression in the PBMCs was detected by microarray comparatively with a control group obtained without the supplement. FIG. 1A shows the results of gene ontology analysis of the gene whose expression induction was observed. Immune responses including type-I interferon signaling pathway were induced.

### 1B: Quantitative PCR Analysis of OAS2, IFIT1, IFIT3, ISG15, IDO1, and PD-L1

The microarray analysis revealed that OAS2, IFIT1, IFIT3, and ISG15, which are interferon stimulated genes (ISGs), and IDO1 (indoleamine 2,3-dioxygenase-1) and PD-L1 (programmed death-ligand 1), which relate to immunotolerance induction, were ranked at high positions, and quantitative PCR showed that their expressions increased in PBMCs that were cultured in the presence of rLVRN (1.5 μg/mL) for 24 hours. As a result, it was observed that the expression of these genes was increased by rLVRN (FIG. 1B).

1C: Experiment of rLVRN Absorption Using Beads

For the purpose of negating any effects caused by impurities contaminated during the production process of rLVRN, rLVRN was absorbed with anti-His tag antibody-coupled beads that adsorb His tag protein, and thus, an experiment of inducing ISGs and IDO1 expression in PBMCs was conducted. As a result, through the absorption with the beads, the induction effect of rLVRN was suppressed. Thus, it was supported that not impurities but rLVRN have an effect on ISGs and IDO1 expression induction (FIG. 1C).

1D: Induction of Genes in PBMC Cultured Directly or Indirectly with Swan71 Cell Forced to Express LVRN

Swan71 cells are a cell line established from human extravillous trophoblast cells that express LVRN on the cell membrane, whereas normal monolayer culture conditions cause a loss of LVRN expression. Accordingly, a Swan71 cell line was first generated to forcibly express LVRN on the cell membrane (LVRN-Swan71). The LVRN expression was detected by cell staining, and the expression on the cell surface was measured by flow cytometry (upper part of FIG. 1D). PMBCs were cultured with LVRN-Swan71 cells in a direct manner or in an indirect manner through a chamber membrane for 24 hours. The effect of LVRN on gene expression induction in PBMCs was measured and compared with that in a cultured mock-expressing cell line (Neo-Swan71). In the direct culture, to eliminate the contamination of Swan71 cells during PBMC collection, anti-CD45 antibody-coupled beads were used to isolate PBMCs for analysis. As a result, it was revealed that the gene expression-inducing effect is stronger in direct culture than in indirect culture. It was found that cell membrane-bound LVRN also has a similar effect on PBMCs (lower part of FIG. 1D).

1E: Examination of Gene Induction Effect of Enzyme-Inactivated rLVRN on PBMC

LVRN is a cell membrane-bound peptidase classified as M1 peptidase. Accordingly, for the purpose of examining whether or not the enzyme activity is involved in the gene expression effect, the enzyme activity was inactivated by introducing a mutation into the gluzincin motif, HEXXH (X) 18E motif (415th to 438th amino acids shown in SEQ ID NO: 1), for the Zn binding enzyme active site, and E416Q rLVRN was obtained. Its effect on PBMCs was measured. As a result, it was shown that the enzymatic activity-inactivated LVRN tends to induce expression of IFIT1, IFIT3, ISG15, OAS2, and IDO1 genes in the same manner as the enzymatic activity-activated LVRN (FIG. 1E).

### 2. Mechanism of ISGs Induction in Immune Cell

2A: IFN Production Inducing Ability of rLVRN

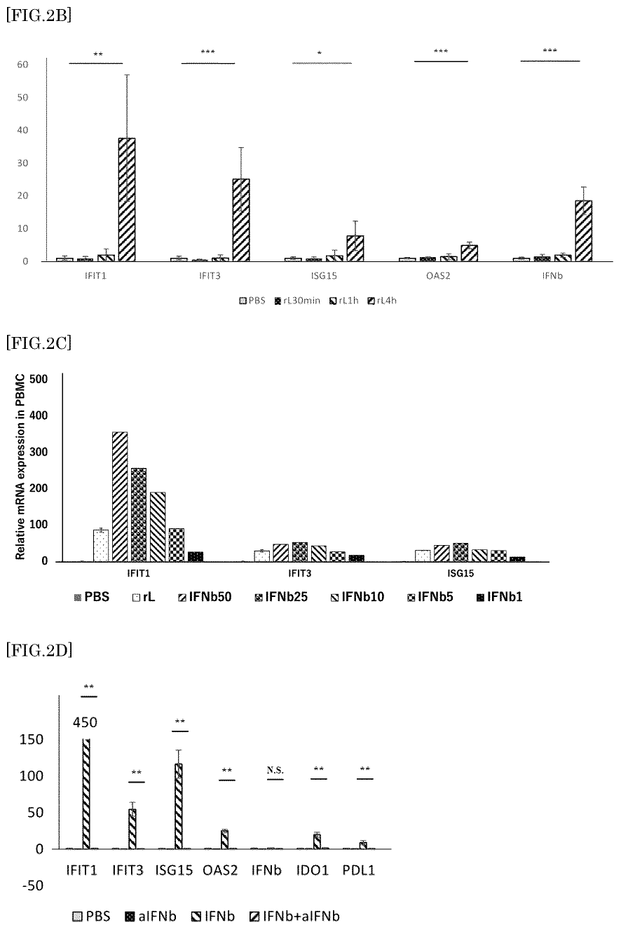

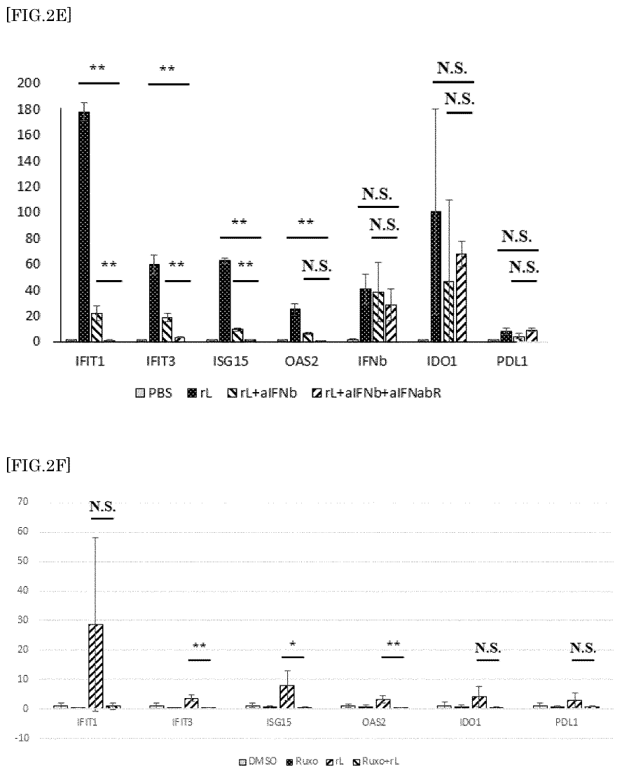

Since the ISGs inductions were detected by the microarray analysis, the induction of IFN production by rLVRN in PBMCs was examined by quantitative PCR. As a result, the production of IFN-β was induced by rLVRN (1.5 μg/mL) in PBMCs, while neither the production of IFN-α nor IFN-γ was induced (FIG. 2A).

2B: Examination of Time Required to Exert the Effect of rLVRN on Gene Induction in PBMC

PBMCs were cultured in the presence of rLVRN (1.5 μg/mL) for 30 minutes, 1 hour, or 4 hours, and the expressions of IFIT1, IFIT3, ISG15, OAS2, and IFN-β were analyzed by quantitative PCR. As a result, the mRNAs of these genes were detected after 4 hours of culture (FIG. 2B).

2C: Determination of Working Concentration of IFN-β Corresponding to rLVRN (1.5 μg/mL) by Titration

PBMCs were stimulated with IFN-β, and the effect on ISGs induction was measured in a concentration-dependent manner. The results were compared with the effect of rLVRN (1.5 μg/mL) on ISGs induction, with the relevant IFN-β concentration set at 10 U/mL (FIG. 2C).

### 2D: Effect of IFN-Neutralizing Antibody on Effect of IFN-β

It was examined whether an IFN-β-neutralizing antibody blocks the induction of the gene expression by IFN-β (100 IU/mL) in PBMCs. As a result, the increase in expression of ISGs, IDO1, and PD-L1 by IFN-β in PBMCs was thoroughly blocked by a neutralizing antibody against IFN-β (FIG. 2D).

2E: Effect of IFN-β Neutralizing Antibody and Type-I IFN Receptor Neutralizing Antibody on the Effect of rLVRN

It was examined whether the induction of the genes by rLVRN (1.5 μg/mL) in PBMCs was blocked by an IFN-β neutralizing antibody or a type-I IFN receptor neutralizing antibody. As a result, the increase in expression of the ISGs induced by rLVRN (1.5 μg/mL), which corresponds to IFN-β (10 IU/mL), was thoroughly blocked by a neutralizing antibody against type-I IFN receptor or a neutralizing antibody against IFN-β, while the increase in expression of IDO1 and PD-L1 was not blocked (FIG. 2E). These results suggest that the induction of the ISGs by LVRN is an effect mediated by the induction of IFN-β production and type-I IFN receptor, and that the effect on the induction of IDO1 expression is derived from a pathway other than an IFN/IFN receptor.

2F: Effect of JAK1,2 Inhibitor, Ruxolitinib, on the rLVRN Effect

A JAK1,2 inhibitor was examined for its effect on the induction of the genes by rLVRN in PBMCs. After PBMCs were cultured with rLVRN (1.5 μg/mL), Ruxolitinib (1 μM), or rLVRN (1.5 μg/mL) and Ruxolitinib (1 μM) for 24 hours, the expression of IFIT1, IFIT3, ISG15, OAS2, IDO1, and PD-L1 was measured by quantitative PCR. As a result, the expression induction of these genes by rLVRN was all inhibited by Ruxolitinib (FIG. 2F). Although a receptor for LVRN has not been identified, this result suggests that JAK1 or JAK2 is involved in the activation.

2G: Comparison of ISG15 and IDO1 Induction Effect by IFN-β and rLVRN

PBMCs from normal non-pregnant subjects (women) (12 cases) were cultured in the presence of IFN-β (10 IU/mL) or rLVRN (1.5 μg/mL) for 24 hours, and the ISG15 and IDO1 induction effects were examined. As a result, IFN-β and rLVRN were different, particularly in the IDO1 induction effect, and it was revealed based on comparison in IDO1/ISG15 ratio that rLVRN has a stronger IDO1 induction effect than IFN-β (FIG. 2G).

3. Analysis of Clinical Samples of Normal Pregnancy, Hypertensive Disorders of Pregnancy, and Pregnancy with Immune Disease
3A: Analysis of Expression of ISG15 and IDO1 in PBMC from Pregnant Women

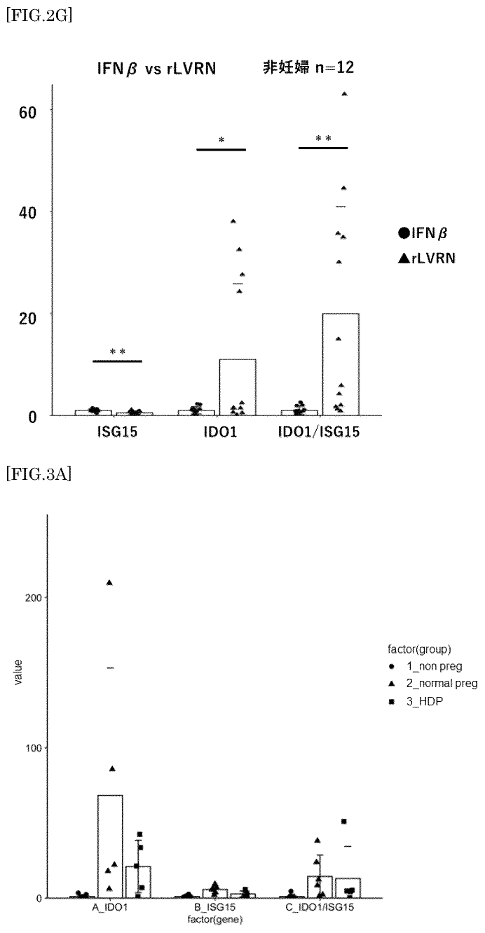

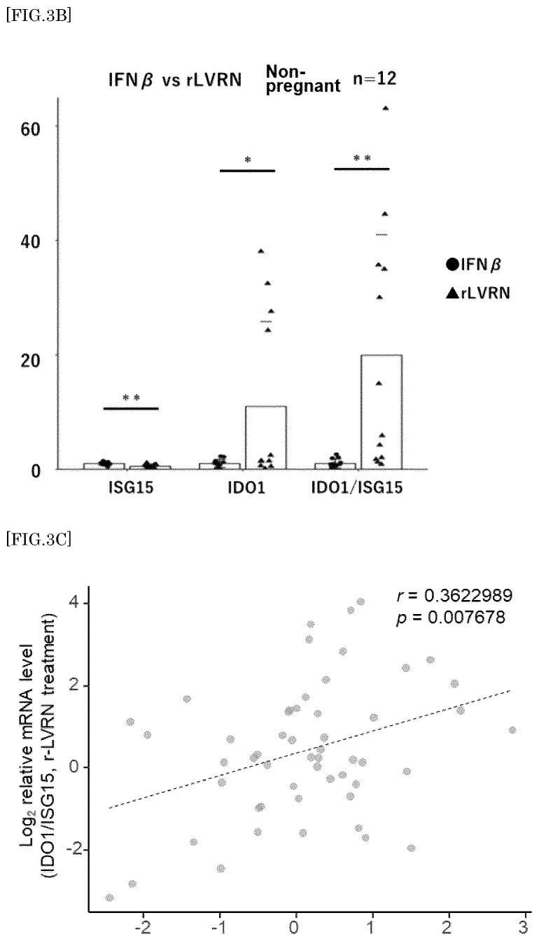

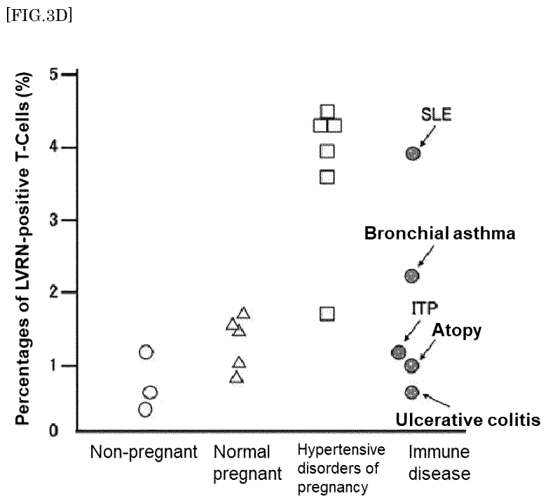

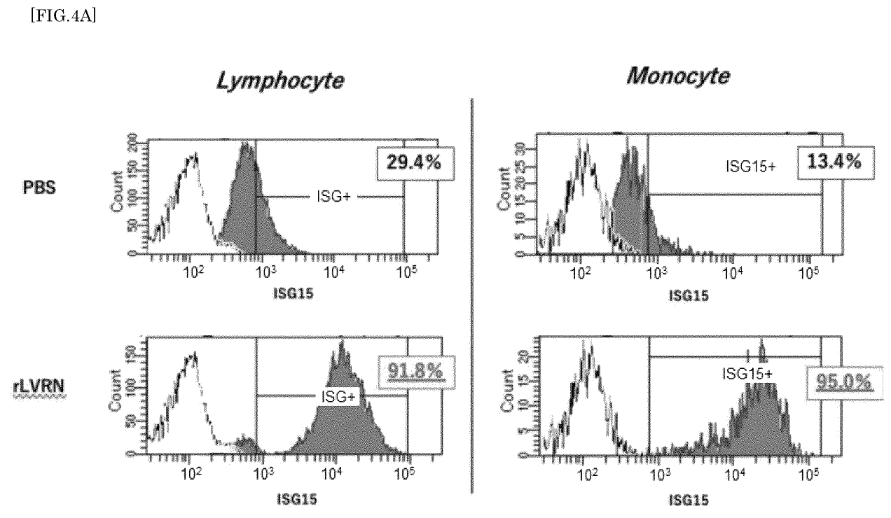

PBMCs from women of normal non-pregnancy (9 cases), normal pregnancy (6 cases), or hypertensive disorders of pregnancy (HDP) (5 cases) were cultured in the presence of rLVRN (1.5 μg/mL) for 24 hours, and the expression of ISG15 and IDO1 was measured by quantitative PCR. As a result of the gene expression comparison, pregnant women exhibited a 5- to 6-fold increase in ISG15, a 100-fold increase in IDO1, and a significantly higher IDO1/ISG15 ratio, compared with non-pregnant women. Thus, this suggests that the IDO1 reaction is further enhanced by pregnancy (FIG. 3A). In addition, women with HDP exhibited a 1/2-fold decrease in ISG15, a 1/6-fold decrease in IDO1, and a decreased IDO1/ISG15 ratio except in one case, compared with normal pregnant women (FIG. 3A).

3B: Effect of IFNβ and rLVRN in PBMC from Normal Pregnant Women

PBMCs from normal pregnant women (29 cases) were cultured in the presence of IFN-β (10 IU/mL) or rLVRN (1.5 μg/mL) for 24 hours, and the expression of ISG15 and IDO1 was measured by quantitative PCR. As a result, IDO1 was induced more strongly by rLVRN than by IFN-β, and the IDO1/ISG15 ratio after the rLVRN stimulation was significantly higher than the IDO1/ISG15 ratio after the IFN-β stimulation (FIG. 3B).

3C: Relation between Growth of Fetus and IDO1/ISG15 Ratio

As an index of growth of a fetus, the standard deviation score (Z score) of a birth weight against gestational age (Itabashi K, Miura F, Uehara R, Nakamura Y. New Japanese neonatal anthropometric charts for gestational age at birth. Pediatr Int. 2014; 56 (5): 702-8.) was used, and the Pearson correlation coefficient (R) was used to analyze linear correlation between fold change of mRNA levels of IDO1 and ISG15 and the Z score of the birth weight. As a result, it was revealed that when the IDO1/ISG15 ratio is high, fetal weight increases, whereas when the ratio is low, fetal weight decreases, suggesting a correlation between the IDO1/ISG15 ratio and fetal weight (FIG. 3C). Accordingly, these suggest that if the IDO1/ISG15 ratio is low, the immunotolerance effect of LVRN is reduced, and placental function is low.

3D: LVRN-Positive T cells in Women of Non-pregnancy, Normal Pregnancy, Hypertensive Disorders of Pregnancy, or Pregnancy with Immune Disease

LVRN-positive T cells from women of non-pregnancy, normal pregnancy, hypertensive disorders of pregnancy, or pregnancy with immune disease were analyzed by flow cytometry. As a result, it was observed that the number of LVRN-positive T cells increases during pregnancy, and is high in hypertensive disorders of pregnancy and systemic lupus erythematosus (SLE) (FIG. 3D).

4. Analysis of Cell in PBMC Reactive to rLVRN
4A: FACS Analysis of ISG15 Protein Expression in PBMC after rLVRN Stimulation

PBMCs were cultured in the presence of rLVRN (1.5 μg/mL) for 24 hours, and the ISG15-expressing cells in lymphocyte fractions and monocyte fractions were analyzed by FACS.

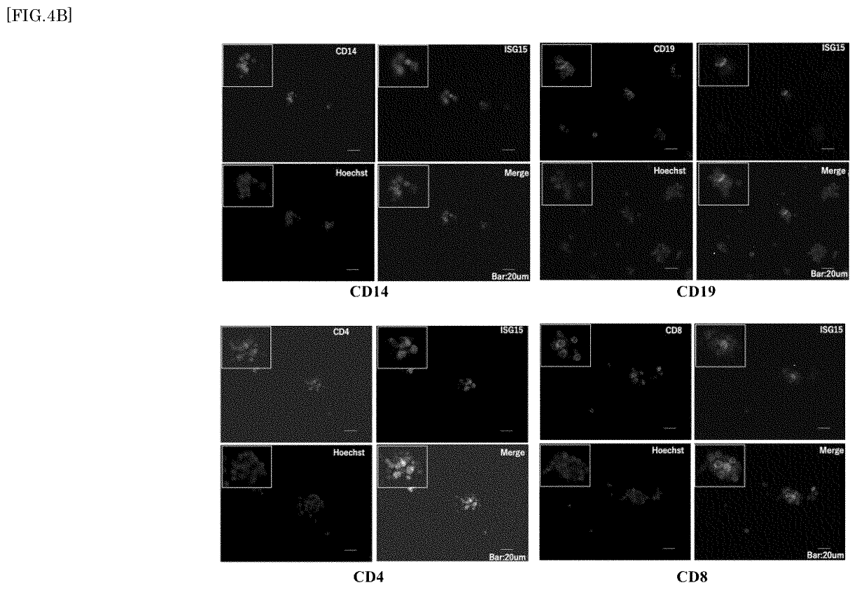

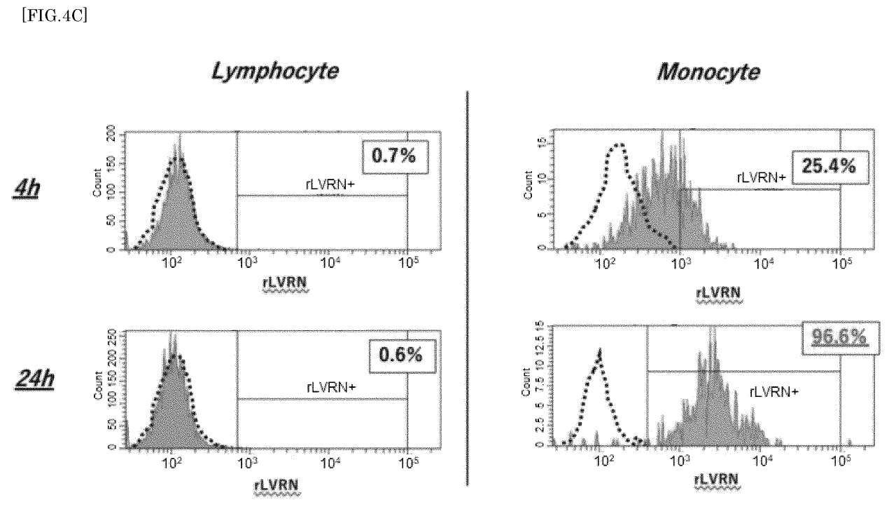

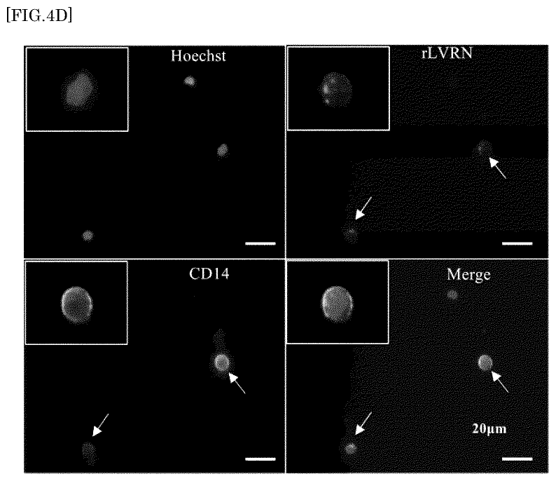

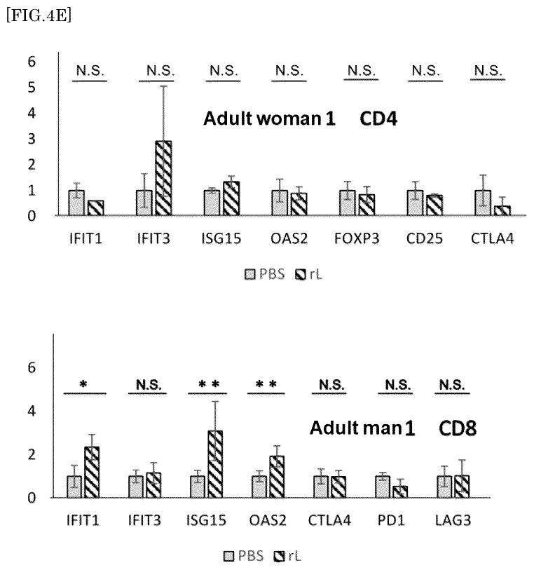

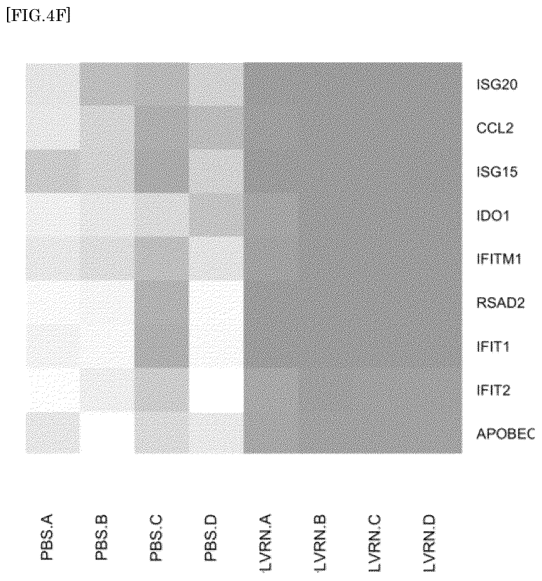

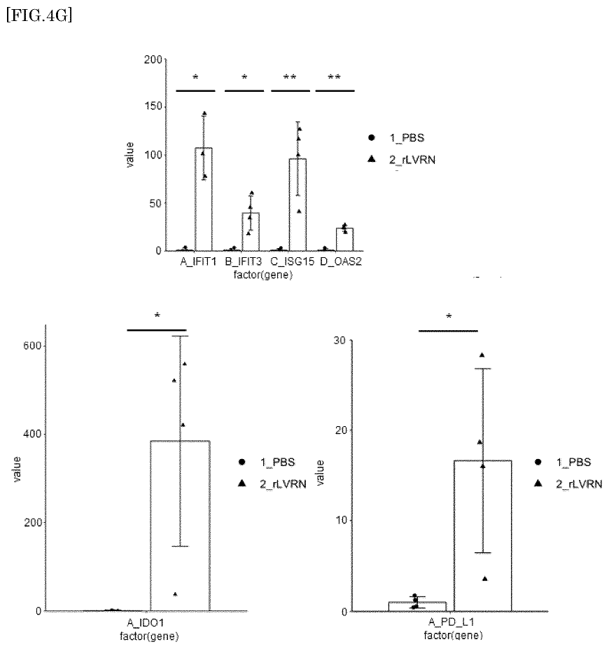

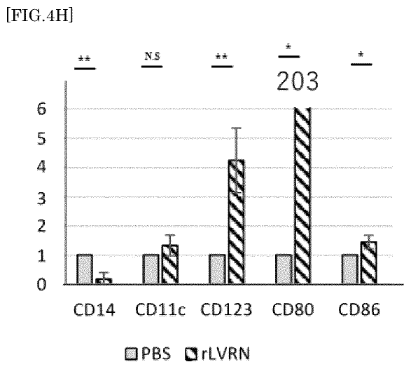

As a result, the expression of ISG15 increased in most of the lymphocyte fractions and monocyte fractions (FIG. 4A).

4B: Analysis, by Cell Staining, of ISG15 Protein Expression in PBMC after rLVRN Stimulation

The expression of ISG15 protein in PBMCs cultured with rLVRN (1.5 μg/mL) for 24 hours was analyzed by cell staining. As a result, the expression of ISG15 protein was observed within cells of CD14 positive monocytes, CD4-positive T lymphocytes, CD8-positive T lymphocytes, and CD19-positive B lymphocytes (FIG. 4B).

4C: FACS Analysis of Labeled rLVRN Binding to or Incorporation into PBMC

PBMCs were cultured for 4 or 24 hours in the presence of rLVRN (1.5 g/mL) labeled with a fluorescent HiLyte555 and analyzed by FACS. The results showed that rLVRN binding to or incorporation into cells was observed in monocyte fractions; detections increased from after 4 hours of culture to after 24 hours, with 96% or more of cells of the monocyte fractions being positive after 24 hours of culture (FIG. 4C). In lymphocyte fractions, rLVRN binding to or incorporation into cells was not observed (FIG. 4C).

4D: Cell Staining Analysis on the Incorporation of Fluorescently Labeled rLVRN into Monocyte Cell

Cells in a monocyte fraction from PBMCs were cultured with fluorescently labeled rLVRN (1.5 g/mL) for 24 hours and analyzed for rLVRN incorporation by cell staining. As a result, fluorescent images show a figure in which the fluorescently labeled rLVRN was incorporated into monocyte cells (FIG. 4D).

4E: Direct Effect of rLVRN on CD4-Positive T Lymphocyte and CD8-Positive T Lymphocyte

CD4-positive fractions and CD8-positive fractions were isolated from PBMCs that were from a normal non-pregnant woman (1 case) and a man (1 case), and the PBMCs were cultured in the presence of rLVRN (1.5 μg/mL) for 24 hours. As a result, the increased level of ISGs expression was low in these lymphocytes. Regarding the significant difference, it varied across samples, suggesting a large individual difference (FIG. 4E). Given that ISG15 protein expression was observed in most lymphocyte fractions, it was suggested that, in primary, the reaction with rLVRN occurs first in CD14-positive monocytes in PBMCs, and then spreads to lymphocytes as a secondary reaction.

4F: Microarray Analysis of a Group of rLVRN-Induced Genes in CD14 Positive Monocyte Cell

CD14-positive monocyte cells were isolated from PBMCs and cultured in the presence of rLVRN (1.5 μg/mL) for 24 hours, and genes expressed were analyzed by microarray. As a result, the reactivity of ISGs was stronger than that of PBMCs by 70-fold and that of IDO1 by 177-fold. In particular, changes in the expression of the following genes were observed.

TABLE 2

| | | |
|---|---|---|
| CD38 | Increase (100 fold) | Maturation marker of human |
| | | monocyte-derived dendritic cell |
| | | Expressed in myeloid-derived |
| | | suppressor cell (MDSC) |
| CD14 | Decrease | Monocyte marker |
| CD80 | Increase by 75 fold | Mature dendritic cell (DC) |
| | (75 fold) | marker |
| 1L27 | Increase | Immunosuppressive cytokine |
| LAG3 | Increase | Immunosuppressive molecule |
| CD274/ | Increase (10 fold) | |
| PD-L1 | | |
| CD9 | Decrease | Necessary for recycle of MHCH of |
| | | DC |
| LILRB1 | Increase | Immunosuppressive molecule |
| APOBEC3A | Increase (both by | Gene editing molecule, presumed to |
| APOBEC3B | 200 or more fold) | have antiviral effect |

4G: Investigating Gene Induction Effect of rLVRN on CD14-Positive Monocyte Cell

CD14-positive monocyte cells were isolated from PBMCs and cultured in the presence of rLVRN (1.5 μg/mL) for 24 hours, and the expression of IFIT1, IFIT3, OSG15, OAS2, IDO1, and PD-L1 was measured by quantitative PCR. As a result, the effect on expression induction of the genes was apparently stronger than in CD4-positive cells and CD8-positive cells (FIG. 4E) was demonstrated (FIG. 4G).

4H: Differentiation Induction (1) of CD14-Positive Monocyte Cell by rLVRN

CD14-positive monocyte cells isolated from PBMCs were cultured in the presence of rLVRN (1.5 μg/mL) for 24 hours, and the expression of CD14, CD11c, CD123, CD80, and CD86 was measured by quantitative PCR. As a result, the expression of CD14, being a monocyte marker, was reduced; that of CD123, being a DC marker, increased; that of CD80, being a DC maturation marker, increased particularly; and that of CD11c remained constant (FIG. 4H). The results suggest that rLVRN induces DC differentiation.

4I: Differentiation Induction (2) of CD14-Positive Monocyte Cell by rLVRN

PBMCs were cultured in the presence of rLVRN (1.5 μg/mL) for 24 hours, and the expression of HLA-DR, CD14, CD11c, and CD123 was analyzed by FACS. As a result, it was suggested that differentiation from CD14-positive cells to CD11c-positive/CD123-positive DCs was induced (FIG. 4I).

5. Effect of rLVRN in THP-1 Cell
5A: Expression of IDO1 in PMA Activated or Inactivated THP-1 Cell after rLVRN Stimulation

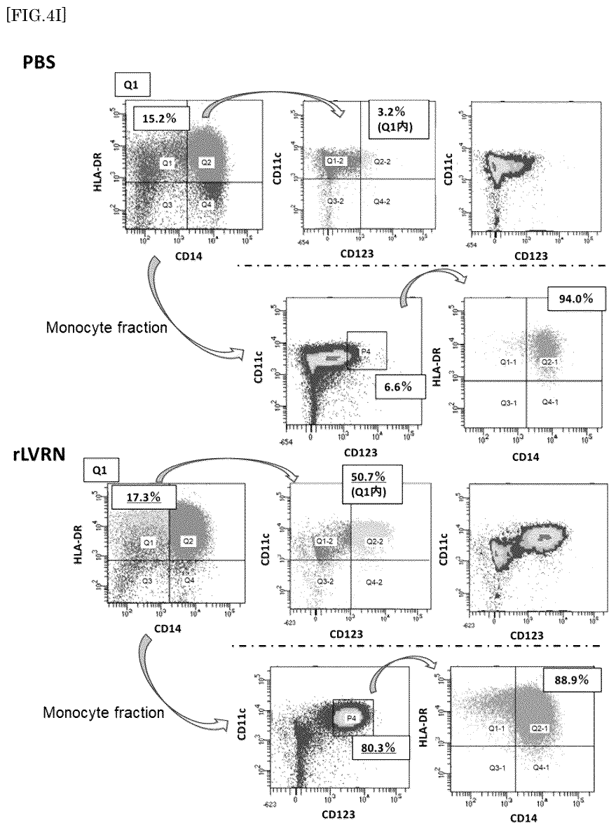

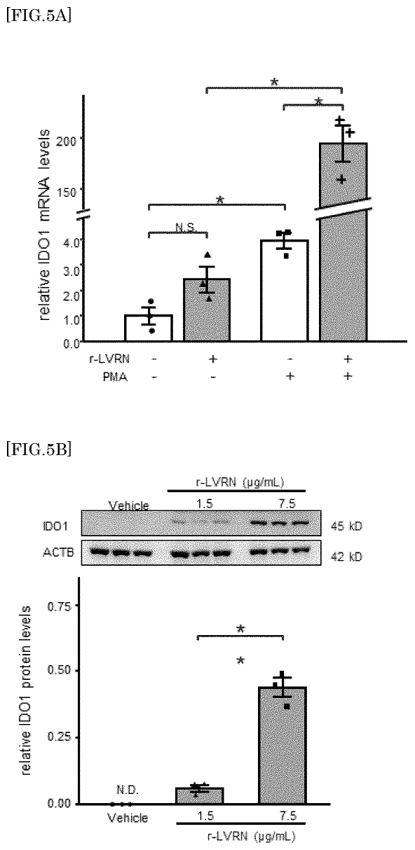

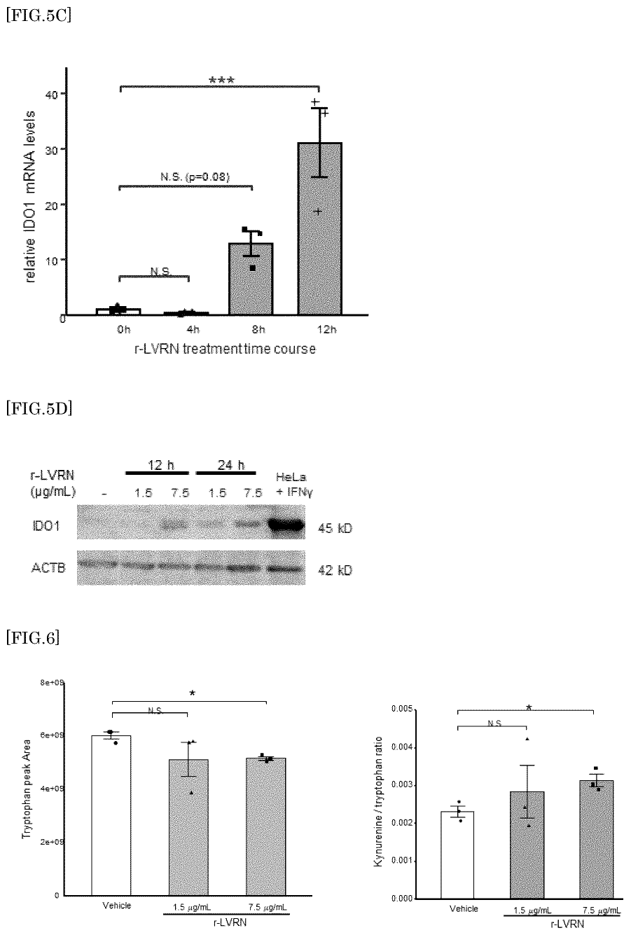

Since the reactivity of CD14-positive monocytes to rLVRN increased after 24 hours of culture, THP-1 cells, a monocyte cell line, were used to evaluate whether activated monocytes increase the reactivity to rLVRN. Inactivated or activated THP-1 cells were treated with PBS or rLVRN (1.5 μg/mL) for 24 hours and measured for the mRNA expression level of IDO1 by quantitative PCR. The rLVRN treatment minimally affected the IDO1 gene induction in the inactivated THP-1 cells (FIG. 5A). On the other hand, in THP-1 cells activated with PMA, the expression of the IDO1 gene was slightly induced, and rLVRN treatment further enhanced the IDO1 expression (FIG. 5A).

5B: Dose-Dependent Expression of IDO1 Protein in PMA-Activated THP-1 Cell after rLVRN Stimulation

A western blot analysis of PMA-activated THP-1 cells treated with PBS or rLVRN (1.5 or 7.5 μg/mL) for 48 hours demonstrates a dose-dependent increase in IDO1 protein with rLVRN treatment (FIG. 5B).

5C: Effect of rLVRN on IDO Induction in PMA-Activated THP-1 Cell

IDO1 mRNA expression levels in PMA-activated THP-1 cells treated with rLVRN (1.5 μg/mL) for 0, 4, 8, or 12 hours were examined by quantitative PCR. rLVRN increased IDO1 gene expression in the activated THP-1 cells after 12 hours of the treatment (FIG. 5C).

5D: Time-dependent Expression of IDO1 Protein in PMA-Activated THP-1 Cell after rLVRN Stimulation

A western blot analysis of PMA-activated THP-1 cells treated with PBS or rLVRN (1.5 or 7.5 μg/mL) for 12 hours or 24 hours demonstrates a time-dependent increase in IDO1 protein through the rLVRN treatment (FIG. 5D).

6. Change of Tryptophan Level and Kynurenine/tryptophan Ratio Caused by rLVRN in PMA-Activated THP-1 Cell

To assess the influences of IDO1 production by rLVRN on the conversion of tryptophan to kynurenine, PMA-activated THP-1 cells were treated with PBS or rLVRN (1.5 or 7.5 μg/mL) for 48 hours, and the medium was evaluated by HPLC-MS for tryptophan level and a kynurenine/tryptophan ratio. Results of the HPLC-MS show that, as compared to a control not treated with rLVRN, the rLVRN treatment allows the tryptophan level in the culture medium from the activated THP-1 cells to be reduced (left part of FIG. 6), and on the other hand, it allows the kynurenine/tryptophan ratio to be increased (right part of FIG. 6). The results suggest that rLVRN promotes tryptophan metabolism, increasing kynurenine and exerting immunosuppressive effects.

SEQ ID NO: 1
Met Gly Pro Pro Ser Ser Ser Gly Phe Tyr Val
Ser His Ala Val Ala Leu Leu Leu Ala Gly Leu
Val Ala Ala Leu Leu Leu Ala Leu Ala Val Leu
Ala Ala Leu Tyr Gly His Cys Glu Arg Val Pro
Pro Ser Glu Leu Pro Gly Leu Arg Asp Ser Glu
Ala Glu Ser Ser Pro Pro Leu Arg Gln Lys Pro
Thr Pro Thr Pro Lys Pro Ser Ser Ala Arg Glu
Leu Ala Val Thr Thr Thr Pro Ser Asn Trp Arg
Pro Pro Gly Pro Trp Asp Gln Leu Arg Leu Pro
Pro Trp Leu Val Pro Leu His Tyr Asp Leu Glu
Leu Trp Pro Gln Leu Arg Pro Asp Glu Leu Pro
Ala Gly Ser Leu Pro Phe Thr Gly Arg Val Asn
Ile Thr Val Arg Cys Thr Val Ala Thr Ser Arg
Leu Leu Leu His Ser Leu Phe Gln Asp Cys Glu
Arg Ala Glu Val Arg Gly Pro Leu Ser Pro Gly
Thr Gly Asn Ala Thr Val Gly Arg Val Pro Val
Asp Asp Val Trp Phe Ala Leu Asp Thr Glu Tyr
Met Val Leu Glu Leu Ser Glu Pro Leu Lys Pro
Gly Ser Ser Tyr Glu Leu Gln Leu Ser Phe Ser
Gly Leu Val Lys Glu Asp Leu Arg Glu Gly Leu
Phe Leu Asn Val Tyr Thr Asp Gln Gly Glu Arg
Arg Ala Leu Leu Ala Ser Gln Leu Glu Pro Thr
Phe Ala Arg Tyr Val Phe Pro Cys Phe Asp Glu
Pro Ala Leu Lys Ala Thr Phe Asn Ile Thr Met
Ile His His Pro Ser Tyr Val Ala Leu Ser Asn
Met Pro Lys Leu Gly Gln Ser Glu Lys Glu Asp
Val Asn Gly Ser Lys Trp Thr Val Thr Thr Phe
Ser Thr Thr Pro His Met Pro Thr Tyr Leu Val
Ala Phe Val Ile Cys Asp Tyr Asp His Val Asn
Arg Thr Glu Arg Gly Lys Glu Ile Arg Ile Trp
Ala Arg Lys Asp Ala Ile Ala Asn Gly Ser Ala
Asp Phe Ala Leu Asn Ile Thr Gly Pro Ile Phe
Ser Phe Leu Glu Asp Leu Phe Asn Ile Ser Tyr
Ser Leu Pro Lys Thr Asp Ile Ile Ala Leu Ser
Ser Phe Asp Asn His Ala Met Glu Asn Trp Gly
Leu Met Ile Phe Asp Glu Ser Gly Leu Leu Leu
Glu Pro Lys Asp Gln Leu Thr Glu Lys Lys Thr
Leu Ile Ser Tyr Val Val Ser His Glu Ile Gly
His Gln Trp Phe Gly Asn Leu Val Thr Met Asn
Trp Trp Asn Asn Ile Trp Leu Asn Glu Gly Phe
Ala Ser Tyr Phe Glu Phe Glu Val Ile Asn Tyr
Phe Asn Pro Lys Leu Pro Arg Asn Glu Ile Phe
Phe Ser Asn Ile Leu His Asn Ile Leu Arg Glu
Asp His Ala Leu Val Thr Arg Ala Val Ala Met
Lys Val Glu Asn Phe Lys Thr Ser Glu Ile Gln
Glu Leu Phe Asp Ile Phe Thr Tyr Ser Lys Gly
Ala Ser Met Ala Arg Met Leu Ser Cys Phe Leu
Asn Glu His Leu Phe Val Ser Ala Leu Lys Ser
Tyr Leu Lys Thr Phe Ser Tyr Ser Asn Ala Glu
Gln Asp Asp Leu Trp Arg His Phe Gln Met Ala
Ile Asp Asp Gln Ser Thr Val Ile Leu Pro Ala
Thr Ile Lys Asn Ile Met Asp Ser Trp Thr His
Gln Ser Gly Phe Pro Val Ile Thr Leu Asn Val
Ser Thr Gly Val Met Lys Gln Glu Pro Phe Tyr
Leu Glu Asn Ile Lys Asn Arg Thr Leu Leu Thr
Ser Asn Asp Thr Trp Ile Val Pro Ile Leu Trp
Ile Lys Asn Gly Thr Thr Gln Pro Leu Val Trp
Leu Asp Gln Ser Ser Lys Val Phe Pro Glu Met
Gln Val Ser Asp Ser Asp His Asp Trp Val Ile
Leu Asn Leu Asn Met Thr Gly Tyr Tyr Arg Val
Asn Tyr Asp Lys Leu Gly Trp Lys Lys Leu Asn
Gln Gln Leu Glu Lys Asp Pro Lys Ala Ile Pro
Val Ile His Arg Leu Gln Leu Ile Asp Asp Ala
Phe Ser Leu Ser Lys Asn Asn Tyr Ile Glu Ile
Glu Thr Ala Leu Glu Leu Thr Lys Tyr Leu Ala
Glu Glu Asp Glu Ile Ile Val Trp His Thr Val
Leu Val Asn Leu Val Thr Arg Asp Leu Val Ser
Glu Val Asn Ile Tyr Asp Ile Tyr Ser Leu Leu
Lys Arg Tyr Leu Leu Lys Arg Leu Asn Leu Ile
Trp Asn Ile Tyr Ser Thr Ile Ile Arg Glu Asn
Val Leu Ala Leu Gln Asp Asp Tyr Leu Ala Leu
Ile Ser Leu Glu Lys Leu Phe Val Thr Ala Cys
Trp Leu Gly Leu Glu Asp Cys Leu Gln Leu Ser
Lys Glu Leu Phe Ala Lys Trp Val Asp His Pro
Glu Asn Glu Ile Pro Tyr Pro Ile Lys Asp Val
Val Leu Cys Tyr Gly Ile Ala Leu Gly Ser Asp
Lys Glu Trp Asp Ile Leu Leu Asn Thr Tyr Thr
Asn Thr Thr Asn Lys Glu Glu Lys Ile Gln Leu
Ala Tyr Ala Met Ser Cys Ser Lys Asp Pro Trp
Ile Leu Asn Arg Tyr Met Glu Tyr Ala Ile Ser
Thr Ser Pro Phe Thr Ser Asn Glu Thr Asn Ile
Ile Glu Val Val Ala Ser Ser Glu Val Gly Arg
Tyr Val Ala Lys Asp Phe Leu Val Asn Asn Trp
Gln Ala Val Ser Lys Arg Tyr Gly Thr Gln Ser
Leu Ile Asn Leu Ile Tyr Thr Ile Gly Arg Thr
Val Thr Thr Asp Leu Gln Ile Val Glu Leu Gln
Gln Phe Phe Ser Asn Met Leu Glu Glu His Gln
Arg Ile Arg Val His Ala Asn Leu Gln Thr Ile
Lys Asn Glu Asn Leu Lys Asn Lys Lys Leu Ser
Ala Arg Ile Ala Ala Trp Leu Arg Arg Asn Thr
SEQ ID NO: 2
atggggcc cccttccagc tcaggcttct atgtgagcca
cgcagtggcc ctgctgctgg ctgggttggt agccgccctc
ctgctggcgc tggccgtact cgccgccttg tacggtcact
gcgagcgcgt cccaccgtcg ctcccctcag gcagaagccg
gagctgcctg gactcaggga ctcggaagcc gagtcttccc
ctcccctcag gcagaagccg acgccgaccc cgaaacccag
cagtgcacgc gagctagcgg tgacgaccac cccgagcaac
tggcgacccc cggggccctg ggaccagcta cgcctgccgc
cctggctcgt gccgctgcac tacgatctgg agctgtggcc
gcagctgagg cccgacgagc ttccggccgg gtctttgccc
ttcactggcc gcgtgaacat cacggtgcgc tgcacggtgg
ccacctctcg actgctgctg catagcctct tccaggactg
cgagcgcgcc gaggtgcggg gacccctttc cccgggcact
gggaacgcca cagtgggccg cgtgcccgtg gacgacgtgt
ggttcgcgct ggacacggaa tacatggtgc tggagctcag
tgagcccctg aaacctggta gcagctacga gctgcagctt
agcttctcgg gcctggtgaa ggaagacctc agggagggac
tcttcctcaa cgtctacacc gaccagggcg agcgcagggc
cctgttagcg tcccagctgg aaccaacatt tgccaggtat
gttttccctt gttttgatga gccagctctg aaggcaactt
ttaatattac aatgattcat catccaagtt atgtggccct
ttccaacatg ccaaagctag gtcagtctga aaaagaagat
gtgaatggaa gcaaatggac tgttacaacc ttttccacta
cgccccacat gccaacttac ttagtcgcat ttgttatatg
tgactatgac cacgtcaaca gaacagaaag gggcaaggag
atacgcatct gggcccggaa agatgcaatc gcaaatggaa
gtgcagactt tgctttgaac atcacaggtc ccatcttctc
ttttctggag gatttgttta atatcagtta ctctcttcca
aaaacagata taattgcctt gtctagtttt gacaaccatg
caatggaaaa ctggggacta atgatatttg atgaatcagg
attgttgttg gaaccaaaag atcaactgac agaaaaaaag
actctgatct cctatgttgt ctcccacgag attggacacc
agtggtttgg aaacttggtt accatgaatt ggtggaacaa
tatctggctc aacgagggtt ttgcatctta ttttgagttt
gaagtaatta actactttaa tcctaaactc ccaagaaatg
agatcttttt ttctaacatt ttacataata tcctcagaga
agatcacgcc ctggtgacta gagctgtggc catgaaggtg
gaaaatttca aaacaagtga aatacaggaa ctctttgaca
tatttactta cagcaaggga gcgtctatgg cccggatgct
ttcttgtttc ttgaatgagc atttatttgt cagtgcactc
aagtcatatt tgaagacatt ttcctactca aacgctgagc
aagatgatct atggaggcat tttcaaatgg ccatagatga
ccagagtaca gttattttgc cagcaacaat aaaaaacata
atggacagtt ggacacacca gagtggtttt ccagtgatca
ctttaaatgt gtctactggc gtcatgaaac aggagccatt
ttatcttgaa aacattaaaa atcggactct tctaaccagc
aatgacacat ggattgtccc tattctttgg ataaaaaatg
gaactacaca acctttagtc tggctagatc aaagcagcaa
agtattccca gaaatgcaag tttcagattc tgaccatgac
tgggtgattt tgaatttgaa tatgactgga tattatagag
ttaattatga taaattaggt tggaagaaac taaatcaaca
acttgaaaag gatcctaagg cgattcctgt tattcacaga
ctgcagttga ttgatgatgc cttttccttg tctaaaaaca
attatattga gattgaaaca gcacttgagt taaccaagta
ccttgctgaa gaagatgaaa ttatagtatg gcatacagtc
ttggtaaact tggtaaccag ggatcttgtt tctgaggtga
acatctatga tatatactca ttattaaaga ggtacctatt
aaagagactt aatttaatat ggaatattta ttcaactata
attcgtgaaa atgtgttggc attacaagat gactacttag
ctctaatatc actggaaaaa ctttttgtaa ctgcgtgttg
gttgggcctt gaagactgcc ttcagctgtc aaaagaactt
ttcgcaaaat gggtggatca tccagaaaat gaaatacctt
atccaattaa agatgtggtt ttatgttatg gcattgcctt
gggaagtgat aaagagtggg acatcttgtt aaatacttac
actaatacaa caaacaaaga agaaaagatt caacttgctt
atgcaatgag ctgcagcaaa gacccatgga tacttaacag
atatatggag tatgccatca gcacatctcc attcacttct
aatgaaacaa atataattga ggttgtggct tcatctgaag
ttggccggta tgtcgcaaaa gacttcttag tcaacaactg
gcaagctgtg agtaaaaggt atggaacaca atcattgatt
aatctaatat atacaatagg gagaaccgta actacagatt
tacagattgt ggagctgcag cagtttttca gtaacatgtt
ggaggaacac cagaggatca gagttcatgc caacttacag
acaataaaga atgaaaatct gaaaaacaag aagctaagtg
ccaggatagc tgcgtggcta aggagaaaca ca

1-25. (canceled)

26. A method for treating a disorder, comprising
administering a composition to a subject in need thereof, wherein
the composition comprises laeverin, laeverin-treated cells, or laeverin-expressing cells, and
the disorder is infertility due to implantation failure, rheumatoid arthritis, glomerulonephritis, or transplant rejection.

27. The method according to claim 26, wherein the composition has an effect of inducing immunotolerance and an effect of inducing interferon stimulated genes.

28. The method according to claim 26, wherein the laeverin is membrane-bound laeverin or soluble laeverin.

29. The method according to claim 26, wherein the laeverin is a polypeptide comprising or consisting of an amino acid sequence having 80% or more, 85% or more, 90% or more, 95% or more, 96% or more, 97% or more, 98% or more, or 99% or more sequence identity to the amino acid sequence shown in SEQ ID NO: 1.

30. The method according to claim 26, wherein the laeverin is a polypeptide comprising or consisting of an amino acid sequence having 80% or more, 85% or more, 90% or more, 95% or more, 96% or more, 97% or more, 98% or more, or 99% or more sequence identity to the amino acid sequence consisting of 65th to 990th amino acids shown in SEQ ID NO: 1.

31. The method according to claim 26, wherein the laeverin-treated cells are immune cells cultured in the presence of laeverin.

32. The method according to claim 26, wherein the laeverin-expressing cells are cells into which at least one gene encoding laeverin are introduced.

33. The method according to claim 26, wherein
the disorder is infertility due to implantation failure,
the composition is administered into the uterus, and
the composition comprises the soluble laeverin or laeverin-treated cells.

34. The method according to claim 26, wherein
the disorder is rheumatoid arthritis,
the composition is administered systemically or locally to the joint, and
the composition comprises the soluble laeverin or laeverin-treated cells.

35. The method according to claim 26, wherein
the disorder is glomerulonephritis,
the composition is administered systemically, and
the composition comprises the soluble laeverin or laeverin-treated cells.

36. The method according to claim 26, wherein
the disorder is transplant rejection in organ transplantation,
the composition is administered systemically, and
the composition comprises the soluble laeverin or laeverin-treated cells.

37. The method according to claim 26, wherein
the disorder is transplant rejection in cell transplantation, and
the composition comprises the laeverin-expressing cells.

38. The method according to claim 26, further comprising
determining the disorder or a risk of onset of the disorder in the subject before the administering the composition to the subject,
wherein
the determining comprises evaluating the effect of inducing immunotolerance and the effect of inducing interferon stimulated genes in cells, treated with laeverin, from the subject.

39. The method according to claim 26, further comprising
determining the disorder or a risk of onset of the disorder in the subject before the administering the composition to the subject,
wherein
the determining comprises comparing, to a reference value, ISG15 expression level, IDO1 expression level, or an IDO1/ISG15 ratio in cells, treated with laeverin, from the subject.

40. The method according to claim 26, further comprising
determining the disorder or a risk of onset of the disorder in the subject before the administering the composition to the subject,
wherein
the determining comprises, to a reference value, an amount or proportion of laeverin-positive T cells in peripheral blood collected from a subject.
